SEQUENCE LISTING (6 sequences)

SEQ ID NO: 1 - protein, 16 aa, Artificial Sequence
  synthetic peptide
RADARADARADARADA

SEQ ID NO: 2 - protein, 18 aa, Artificial Sequence
  synthetic peptide
RARADADARARADADADA

SEQ ID NO: 3 - protein, 12 aa, Artificial Sequence
  synthetic peptide
KLDLKLDLKLDL

SEQ ID NO: 4 - protein, 8 aa, Artificial Sequence
  synthetic peptide
FKFEFKFE

SEQ ID NO: 5 - protein, 16 aa, Artificial Sequence
  synthetic peptide
AEAEAKAKAEAEAKAK

SEQ ID NO: 6 - protein, 8 aa, Artificial Sequence
  synthetic peptide
FEFEFKFK

(19) United States
(12) Patent Application Publication
(10) Pub. No.: US 2023/0392123 A1
(43) Pub. Date: Dec. 7, 2023

(54) SPHEROIDAL SELF-ASSEMBLED PEPTIDE HYDROGELS COMPRISING CELLS

(71) Applicants: KATHOLIEKE UNIVERSITEIT LEUVEN, Leuven (BE); TAMPERE UNIVERSITY FOUNDATION SR, Tampere (FI)

(72) Inventors: Mostafa KIAMEHR, Leuven (BE); Catherine VERFAILLIE, Leuven (BE)

(21) Appl. No.: 18/249,174

(22) PCT Filed: Oct. 15, 2021
(86) PCT No.: PCT/EP2021/078668

§ 371 Date: Apr. 14, 2023

(30) Foreign Application Priority Data
Oct. 15, 2020 (FI) 20206018

(51) Int. Cl. C12N 5/071; C12N 5/077

(52) U.S. Cl. CPC C12N 5/0671; C12N 5/0657; C12N 2513/00; C12N 2506/45; C12N 2533/90; C12N 2503/02

(57) ABSTRACT

The invention relates to self-assembled peptide hydrogel spheroids, with a diameter of between 500 and 2500 μm, comprising cells encapsulated within said hydrogel. The invention further relates to in vitro methods of producing a hydrogel spheroid comprising cells, the method comprising: a) mixing a suspension of cells with a self-assembling peptide, and b) transferring an aliquot of the mixture obtained in step a) into an aqueous salt solution by applying a droplet of the mixture to the surface of the solution thereby forming a hydrogel spheroid comprising encapsulated cells, wherein the droplet has a volume of between 0.1 and 20 μl. and wherein the droplet comprises cells at a concentration of between 1×10⁵ to 5×10⁷ cells per ml solution.

### FIELD OF THE INVENTION

The present invention relates generally to the field of medicine, more specifically to the fields of drug development, disease modelling, tissue engineering and regenerative medicine. The present invention provides hydrogel spheroids composed of cells encapsulated within a self-assembled peptide hydrogel scaffold and to a method for the preparation of these hydrogel spheroids.

### BACKGROUND OF THE INVENTION

Before a new drug can enter the market, it must go through several stages of a long and difficult drug development process in order to result in a new drug that is safe, efficacious and meets all regulatory requirements. However, despite extensive testing, some severe adverse drug reactions can present only in the late clinical stages of drug development or even post marketing, resulting in costly drug failure and decreased patient safety. Hepatotoxicity and cardiotoxicity are the most frequent causes of late stage drug failures.

Test systems that accurately predict organ toxicity of candidate drugs are important tools for preventing drug failure only in the late phases of drug development. Historically, toxicology studies have relied heavily on animal models, the predictive power of which is, however, limited due to substantial inter-species differences, for example, in drug absorption, metabolism and excretion.

In contrast to animal studies, in vitro assays, which utilize human cells, represent potential models for investigating human-specific toxicity profiles. Indeed, primary hepatocytes have been considered the gold standard model for predicting drug-induced liver injury. However, when cultured as 2D monolayers, primary hepatocytes dedifferentiate and lose crucial hepatic functions within hours after plating. Since it is of fundamental importance that the culture system supports the long-term maintenance of relevant cellular phenotypes, dedifferentiation of the primary hepatocytes severely limits their potential as a suitable system for predicting human drug metabolism and drug toxicity.

To prevent the dedifferentiation of primary hepatocytes in vitro, a variety of methods has been developed. For example, overlaying 2D cultures of primary hepatocytes with a thin layer of extracellular matrix (ECM) proteins mimics the physiological microenvironment of hepatocytes in vivo improving the stability of the hepatocytes. However, since dedifferentiation still remains, such sandwich cultures are primarily used for short-term studies. Dedifferentiation can be reduced further, thus enabling extended culture times up to 14 days, by renewing the ECM overlay every 3-4 days. Spheroids and organoids are both 3D structures made of many cells. Spheroids are simple self-aggregating clusters of cells that form spontaneously when adherent cells are denied an attachment surface, whereas organoids are complex multicellular structures that self-organize into microscopic versions of parent organs when given a scaffolding extracellular environment. Primary hepatocytes cultured as hepatic spheroids generated by gravitational aggregation in hanging-drops or on ultra-low attachment surfaces have been reported to remain phenotypically and functionally stable over at least 5 weeks in culture and maintain endogenous hepatic functions, such as albumin and urea production as well as glycogen storage [Bell et al. (2018) Toxicol Sci 162(2), 655-666]. Hepatic organoids derived from iPSCs have been developed by combining hepatic progenitor cells with human umbilical vein endothelial cells and human mesenchymal stem cells.

Despite improvements allowing prolonged culturing without dedifferentiation, primary hepatocytes are not perfect for toxicity studies because they are still difficult to acquire, and most importantly, display functional variability between donors. One potential solution is to differentiate pluripotent stem cells toward a hepatic fate. Indeed, recent advances in the production of not only stem cell-derived hepatocytes but also, for example, cardiomyocytes make stem cells an ideal source of cells for large-scale screening tests, in both 2D and 3D, and an attractive alternative to current industrial screening models. One of the main advantages associated with induced pluripotent stem cells is that they can proliferate indefinitely and with careful handling maintain relatively stable genomic transcriptional and epigenetic profiles.

Compared to 2D culture, 3D culture more closely mimics the in vivo tissue where cells are able to communicate in multidimensions and form biochemical and physico-chemical gradients within their secreted extra cellular matrix (ECM). ECM enables the cells to freely migrate within the construct and form their desired microenvironment similar to the condition in the native tissue. In such a system, cells can reorganize and create a polarized arrangement which is particularly important for hepatocytes. This is while in 2D, the cells can only be polarized partially. Therefore, a 3D environment can improve cell viability, migration, cellular content, organization and polarization and subsequently functionality of the cultured tissue type.

Using iPSCs hold several advantages over using primary human cells. iPSCs are an inexhaustible source of cells, which will circumvent the limitation and scares availability of the primary human cells. Most importantly, existing primary cultures are suffering from inter-donor and inter-batch variability and using iPSC-derived cells could eliminate this limitation. Additionally, a single iPSC line can be differentiated to various and multiple tissue types all sharing similar genetic information. iPSCs could be also generated from any individual which provides the opportunity of studying a certain disease or the effect of genetic factors in a patient of interest opening the avenue for developing personalized medicine.

Despite improvements in drug screening technologies and promise in recent advances, there is still a need for accurate predictive models of human toxicity which aim at faster failing of poor drugs, thereby delivering safer and more efficacious medicines for the patient.

WO2004007683 discloses the formation of hydrogels wherein a mixture of cells and self-assembling peptides are loaded into multiwells and culture medium is subsequently added. This leads to a irregularly shaped hydrogel with heterogeneous cell aggregates throughout the hydrogel.

Hainline et al. (2019) Macromol. Biosci. 19(1), e1800249 discloses, methods wherein self-assembling peptides and cells are injected in a medium which leads to cell clusters in a string of polymerized hydrogel.

Chiu et al. (2014) Nanomed. Nanotech. Biol. Med. 10(5), 1065-1073, discloses methods wherein an already formed peptide hydrogel is immersed in complete a medium containing cardiomyocytes, for subsequent attachment to the hydrogel.

US2004242469 discloses methods wherein cells are seeded on top of a self-assembled hydrogel that contains no cells.

Song et al. (2020) J. Nanobiotech 18(1), 90, discloses methods wherein a medium with self-assembling peptides and cells are applied to the bottom of culture plate, whereafter medium is added to generate a hydrogel.

Corning Puramatrix™ peptide hydrogel instruction leaflet discloses methods wherein a 15 μl volume droplet with self-assembling peptides and cells (2.5×10⁵ to 5×10⁵ cells/ml) are applied at the side of a well with medium. Upon contact with the medium a hydrogel droplet with an irregular shape is formed. This method is used as a quick screen for optimizing conditions for encapsulating a cell type of interest. The encapsulated cells can be tested for viability, morphology and immunostaining.

### SUMMARY OF THE INVENTION

An object of the present invention is to provide means and methods for drug development, disease modelling and regenerative medicine so as to overcome problems associated with conventional cell spheroids and organoids. This object is achieved by a hydrogel spheroid comprising cells, a method of producing the same, assays utilizing the same and a kit comprising the same, which are characterized by what is stated in the independent claims. Preferred embodiments of the invention are disclosed in the dependent claims.

Accordingly, the invention provides a hydrogel spheroid comprising cells encapsulated within a spheroidal scaffold of a self-assembled peptide hydrogel.

The invention also provides an in vitro method of producing these hydrogel spheroids comprising cells, wherein the method comprises:
a) mixing a suspension of cells with a self-assembling peptide, and
b) transferring an aliquot of the mixture obtained in step a) into an aqueous salt-containing solution to induce self-assembly of a hydrogel spheroid which encapsulates the cells, thereby forming a hydrogel spheroid comprising cells.

Also provided is a hydrogel spheroid comprising cells obtainable by the above-mentioned method.

In a further aspect, the invention provides an assay for identifying a candidate compound for drug development, wherein the method comprises:
i. contacting the hydrogel spheroid comprising cells with a test compound,
ii. detecting whether the test compound has a pharmacological effect on the hydrogel spheroid comprising cells, and
iii. identifying the test compound as a candidate compound for drug development if the detected pharmacological effect is the desired pharmacological effect.

Also provided is an assay for identifying a candidate drug for the treatment of a disease, comprising:
i. contacting the hydrogel spheroid comprising cells showing a disease phenotype of interest with a test compound,
ii. detecting whether the test compound has an effect on the cells in the hydrogel spheroid, and
iii. identifying the test compound as a candidate drug for the treatment of the disease, if the detected effect is a positive pharmacological effect associated with amelioration of the disease.

Furthermore, the invention provides an assay for determining toxicity of a test compound, comprising:
i. contacting the hydrogel spheroid comprising cells with the test compound,
ii. assessing whether the test compound has an effect on the cells in the hydrogel spheroid, and
iii. determining the toxicity of the test compound based on the assessed effect.

In a further aspect, the invention provides a kit for producing the hydrogel spheroid comprising cells, the kit comprising:
a) a self-assembling peptide composition, and
b) one or more components selected from the following:
   i) additional peptides and/or proteins to create a favourable environment for growth and differentiation of the cell type of interest,
   ii) an aqueous salt-containing solution to induce self-assembly of a hydrogel spheroid,
   iii) cells to be encapsulated in the hydrogel spheroid to be produced,
   iv) one or more enzymes required for cellular detachment prior to hydrogel spheroid generation,
   v) an optimized isotonic non-salt solution in which the cells are to be mixed with the self-assembling peptide composition,
   vi) optimized medium including essential growth factors and small molecules for differentiation of the cells to be encapsulated within the hydrogel spheroids towards cell or tissue types of interest, and
   vii) optimized medium including essential growth factors for a long-term culture and maintenance of the hydrogel spheroids comprising to be produced.

The present invention shows that hepatic progenitor cells can become mature in the hydrogel spheroids and keep their phenotype during at least 4 weeks of culture. The present invention shows shows that dividing cells can grow and contribute to create a denser environment in the hydrogel spheroids, compared to 2D cultures.

The present invention shows that the expression of key genes of hepatocytes is higher in a hydrogel spheroid compared to a 2D culture showing superiority of hydrogel spheroids for the growth of hepatic cells.

The present invention shows that beating cardiomyocytes remain their phenotype and beating in a co-culture with hepatic cells in a hydrogel spheroid. This allows studying both tissue types in a single environment, which is particularly suitable for organ-on-chip projects or drug metabolism and toxicity studies.

The present invention shows that various combinations of extracellular matrix components can be added to the hydrogel spheroids.

This allows tailoring hydrogel spheroids according to the desired cell type(s) as mono- or co-culture with other cell types.

The present invention demonstrates the successful co-culture of iPSC-hepatocytes, macrophages and endothelial cells in hydrogel spheroids.

The present invention demonstrates the successful co-culture of iPSC-hepatocytes, macrophages, stellate cells, and endothelial cells in hydrogel spheroids.

The present invention illustrate the co-culture of hepatocytes together with non-parenchymal cells in hydrogel spheroids.

The invention is further summarized in the following statements:
1. A self-assembled peptide hydrogel spheroid, with a diameter of between 500 and 2500 μm, comprising cells encapsulated within said hydrogel.
2. The hydrogel spheroid according to statement 1, which is suspended in a cell culture medium.
3. The hydrogel spheroid according to statement 1 or 2, wherein the diameter of the spheroid is between 500 μm and 2000 μm or between 900 μm and 2000 μm.
4. The hydrogel spheroid, comprising between 1×10⁶ cells/ml and 5×10⁷ cells/ml.
5. The hydrogel spheroid according to any one of statements 1 to 4, wherein said cells are evenly distributed throughout the hydrogel spheroid.
6. The hydrogel spheroid according to any one of statements 1 to 4, wherein said cells occur as a layer of cells at the periphery of the hydrogel spheroid.
7. The hydrogel spheroid according to any one of statements 1 to 6, further comprising one or more extracellular matrix proteins or peptides.
8. The hydrogel spheroid according to any one of statements 1 to 7, wherein said cells are selected from the group consisting of hepatic cells including hepatocytes and non-parenchymal cells (NPCs), cardiac and muscle cells, endothelial cells, neural cells, pancreatic cells, osteocytes, chondrocytes, intestinal cells, fibroblasts, adipocytes, epithelial cells, pituitary cells, renal cells, lung cells, secretory cells, oral cells, germ cells, and cancer cell types or a mixture thereof.
9. The hydrogel spheroid according to any one of statements 1 to 8, wherein the cells are hepatic cells.
10. The hydrogel spheroid according to statement 8 or 9, wherein the hepatic cells are selected from the group consisting of primary hepatocytes, cells of a hepatic cell line, hepatic progenitor cells derived from mesenchymal or pluripotent stem cells, and hepatocytes differentiated from hepatic progenitor cells or mesenchymal or pluripotent stem cells.
11. The hydrogel spheroid according to statement 8, 9 or 10, wherein the hepatic cells are hepatocytes differentiated from pluripotent stem cells.
12. The hydrogel spheroid according to any of statement 8 to 11, wherein the hepatic cells express ALB, ASGR1 and AFP, NTCP, CYP3A4, CK18, MRP2, PEPCK, AAT1 and occludin.
13. The hydrogel spheroid according to statement 8, wherein the cardiac cells are selected from the group consisting of primary cardiomyocytes, cells of a cardiomyocyte cell line and cardiomyocytes differentiated from pluripotent stem cells.
14. The hydrogel spheroid according to statement 13, wherein the cardiac cells are primary cardiomyocytes.
15. The hydrogel spheroid according to any one of statements 1 to 14, wherein the cells are co-culture of hepatocytes and cardiomyocytes, typically beating cardiomyocytes.
16. An in vitro method of producing a hydrogel spheroid comprising cells, the method comprising: a) mixing a suspension of cells with a self-assembling peptide, and b) transferring an aliquot of the mixture obtained in step a) into an aqueous salt solution by applying a droplet of the mixture to the surface of the solution thereby forming a hydrogel spheroid comprising encapsulated cells,
wherein the droplet has a volume of between 0.1 and 20 μl, and wherein the droplet comprises cells at a concentration of between 1×10⁵ to 5×10⁷ cells per ml solution.
17. The method according to statement 16, wherein the volume of the aliquot ranges from about 0.5 μl to about 20 μl.
18. The method according to statement 16 or 17, wherein said mixture comprises cells at a concentration of between 1×10⁶ cells per ml solution.
19. The method according to any one of statements 16 to 18, wherein the cells are hepatic cells.
20. The method according to statement 19, wherein the hepatic cells are hepatocytes differentiated from pluripotent stem cells.
21. The method according to any one of statements 16 to 20, wherein the aqueous salt solution is a cell culture medium, preferably a differentiation medium for mesenchymal or pluripotent stem cells or progenitor cells.
22. The method according to any one of statements 16 to 21, further comprising cultivating the hydrogel spheroid in a cell culture medium for at least 4?, 8, 15, 20, 30 or 40 days.
23. The method according to any one of statements 16 to 22, wherein the hydrogel spheroids are suspended in said culture medium.
24. The method according to any one of statements 16 to 23, further comprising a step of freezing the hydrogel spheroids comprising cells.
25. Use of hydrogel spheroids comprising cells according to any one of statements 1 to 15, or prepared by the method according to any one of statements 16 to 24, for toxicity testing of compounds or pharmaceutical activity.
26. Hydrogel spheroids comprising cells according to any one of statements 1 to 15, or prepared by the method according to any one of statements 16 to 24, for use as a medicament.

Further aspects, embodiments and details are set forth in following figures, detailed description, examples, and dependent claims.

### BRIEF DESCRIPTION OF THE DRAWINGS

The accompanying drawings illustrate several embodiments of the disclosed subject matter, and together with the description, serve to explain principles of the disclosed compositions and methods.

FIG. 1A is a schematic representation of a method for differentiating iPSC-derived hepatic cells in a conventional 2D culture or in 3D spheroids of the invention. FIG. 1B shows a cartoon illustration of a method for generating hydrogel spheroids comprising hepatic cells using a self-assembling peptide hydrogel (PuraMatrix™) When the pipette tip approaches and touches the medium surface, the droplet is released from the pipette, sinks in the medium and forms a hydrogel spheroid.

FIG. 2 demonstrates that hepatic cells in 2D culture lose their typical morphology and phenotype relatively fast. The panel shows bright field images of hepatocytes derived from two iPSC cell lines (iPSC-1 and iPSC-2) in 2D culture at days 8, 14, and 26 after switching to the maturation medium. Scale bar: 400 μm.

FIG. 3A shows bright field images of iPSC-derived hepatic progenitor cells (iPSC-1 or iPSC-2) in 2D culture before being collected for generating hydrogel spheroids. Scale bar: 400 μm. FIG. 3B is a view of a hydrogel spheroid comprising hepatc cells derived from iPSC-1 at day 8 of the culture captured under Binocular Stereo Microscope. FIG. 3C shows bright field images of a hydrogel spheroid comprising hepatic cells derived from iPSC-2 during the 26 days of hepatic culture in maturation medium at time points days 4, 8, 20 and 26. Scale bar: 1 mm.

FIG. 4A shows results of a live/dead assay at day 7 for hydrogel spheroids comprising hepatic cells derived from iPSC-1, while FIG. 4B shows the results at days 1 and 12 for hydrogel spheroids comprising hepatic cells derived from iPSC-2. In FIG. 4A, the lower row shows a higher magnification of the dashed rectangle in the upper row. Images were captured under Evos inverted fluorescence microscope. Scale bars: 4A, upper row 1 mm, lower row: 400 μm. 4B, upper row 400 μm, lower row 1 mm.

FIG. 5A shows representative bright field images of hydrogel spheroids comprising HepG2 cells showing the growth of cells in the hydrogel spheroids over time in the culture. FIG. 5B shows results of live/dead assays from representative hydrogels spheroids comprising hepatocytes on days 6 and 13, demonstrating that the majority of HepG2 remained viable during their culture in hydrogel spheroids. Scale bars: 5A, 1 mm; 5B, upper row: 1 mm; lower row: 400 μm.

FIG. 6A shows immunostaining of iPSC-derived hepatocytes in 2D culture, whereas FIGS. 6B and 6C show immunostaining of 3D cell growth in hydrogel spheroids. FIGS. 6A and 6B: hepatocytes derived from iPSC-1; FIG. 6C: hepatocytes derived from iPSC-2. FIG. 6A shows the immunostaining for AFP and ALB in 2D culture. FIG. 6B shows the immunostaining for AFP, ALB, and Ki67 at day 8 and day 26, and ASGR1 at day 8. FIG. 6C shows the immunostaining for AFP and ALB at day 12. Lower row is a zoomed view of the upper row. Nuclei was stained by DAPI. Scale bar on all panels: 200 μm.

FIG. 7 shows immunostaining of hydrogel spheroids comprising HepG2 cells on day 23 of culture showed positive for hepatic markers AFP, ALB, and ASGR1. Nuclei was stained by DAPI. Scale bars: upper row, 1 mm; lower row, 200 μm.

FIG. 8 shows Haematoxylin and Eosin (H&E) staining of representative hydrogel spheroids comprising hepatic cells on days 8 and 26. Images on the right panel are zoomed views of the dashed rectangle shown on the left. The black arrow on the right lower panel shows a dense area of cells formed with biliary epithelium morphology within the hydrogel spheroids.

FIG. 9 shows H&E staining of hydrogel spheroids comprising hepatic cells on day 13. The image on the right shows a higher magnification of the dashed rectangle area shown in the image on the left.

FIG. 10 illustrates immunohistochemistry results of representative hydrogel spheroids comprising hepatic cells at days 8 and 26 for markers AFP, CK19. Nuclei was stained by DAPI. Scale bars: day 8, 200 μm; day 26, upper row: 1 mm; lower row: 200 μm.

FIG. 11 illustrates immunohistochemistry results of hydrogel spheroids comprising HepG2 spheroids on day 13 for the markers AFP and ALB. Nuclei was stained by DAPI. Scalebar: 200 μm.

FIG. 12 shows comparative qPCR analysis of hepatic spheroid at days 8 and 26 (iPSC-1), and days 8, 20, and 26 (iPSC-2) in comparison to their 2D counterparts as well as HepG2 (passage 5, day 5) and PHHs (days 1 and 2). FIG. 12A represents key stem cell-(OCT4) and hepatic-(FOXA2, AFP, ALB) specific genes. FIGS. 12B, and C represents key genes important in hepatic functionality. The values are relative to iPSCs at pluripotent stage, and GAPDH has been used as housekeeping gene to normalize the values.

FIG. 13 shows a bright filed image of a hydrogel spheroid with co-cultured iPSC-derived cardiomyocytes (large aggregate, white arrow) and hepatocytes in maturation medium on day 1 of the culture. Scale bar: 1 mm.

FIG. 14 shows immunostaining of iPSC-derived cardiomyocytes and hepatocytes co-cultured in hydrogel spheroids on days 8 and 26 for cardiac marker Troponin T (T. T), and hepatic markers APF and CK19. Nuclei was stained by DAPI. Scale bar: 200 μm.

FIG. 15. A) Represents the morphology of hydrogel spheroids with hepatic mono-cultures derived from iPSC-3 generated in three different sizes (2 ul, 3.5 ul, and 7 ul) captured by digital camera (top raw) and under brightfield microscope (lower raw). B) Comparative qPCR analysis of hydrogel spheroid with hepatic mono-cultures (after 32 days in 3D culture) in 4 different sizes (1 ul, 2 ul, 3.5 ul, and 7 ul) derived from iPSC-3 and their comparison to freshly isolated PHHs from two individual donors (F108 and F125), 2D HepG2 (passage 12) as well as 2D iPSC-3 derived hepatocytes. The delta Ct values were calculated relative to the expression of RPL19. Each bar for 3D and 2D iPSC-derived hepatocytes represents the average expression from 2-4 replicates, each replicate contained three individual hydrogel spheroids. All replicates were derived from one hepatic differentiation.

FIG. 16. Comparative qPCR analysis of hydrogel spheroids with hepatic mono-cultures generated in panel A for the key hepatic markers of CYP3A4, NTCP, HNF4, HNF6, PEPCK, CYP1A2, CYP2C9, CYP2D6, ALB, AFP. The delta Ct values were calculated relative to the expression of RPL19. Each bar represents the average expression from 4 replicates, each replicate contained minimum three individual hydrogel spheroidals pooled together. All replicates were derived from one hepatic differentiation.

FIG. 17. A) Production of hydrogel spheroids in high quantities. The image on the left shows the bulk culture of hydrogel spheroids with cells in a spinning flask. The mid images show the representative spheroidal 3D cultures in spinning flask after 2, 6, and 12 days in culture captured under brightfield microscope. The image on the right is a representative H&E staining of spheroidal 3D cultures collected from the spinning flask after 12 days of culture. The results presented in this figure are from a single experiment. B) hydrogel spheroids with HepG2 mono-cultures in high-throughput format handled by a robotic automation platform. The panel on the left shows hydrogel spheroids with HepG2 cells in culture in 384 well plate format. Each well contains one hydrogel spheroid structure. The zoom in image shows one of the spheroidal structures after 96 hours in culture. The image on the right is a representative panel of hydrogel spheroids with HepG2 cells on the left treated by live (green)/dead (dead) staining after 19 days of culture imaged by a high-content analysis system. Nuclei were stained by Hoechst. The hydrogel spheroids with cells on the last column on the right were treated with a toxic agent (0.1% Triton X) resulting in cell death visible in red colour.

FIG. 18. Co-culture of iPSC-3 derived hepatocytes (Sigma HC3X) and endothelial cells (ETV2-Spi1), termed as HE cultures, as hydrogel spheroids with cells obtained by self-assembling peptides. A) Image on the left, shows the hepatic progenitor cells in 2D. Image on the right shows the endothelial progenitor cells in 2D. Images were captured with 10× magnification under brightfield microscope. B) Demonstrates spheroidal HE co-cultures at 2, 12, and 31 days in culture after generation. C) Confocal imaging from a hydrogel spheroid with a co-culture of cells at day 32 for endothelial (CD31, green) and hepatic (PEPCK, red) markers demonstrates interconnected vasculature networks within the spheroidal cultures. Scale bar=200 μm. D) Top left two images show a representative H&E staining of the sectioned hydrogel spheroid co-culture (day 12). Immunohistochemistry of the sectioned hydrogel spheroid with HE co-culture (day 12) for the hepatocyte (NTCP, CYP3A4, CK18, MRP2, PEPCK, and Occludin) and non-parenchymal (CK7, CDH5, CD31, aSMA, and PDGFRb), as well as ER stress (GORASP2 and PCK1) and early apoptotic (ACASP3) markers. The images of representatives of minimum three hydrogel spheroids all driven from one differentiation. Scale bar=50 μm.

FIG. 19. A) Co-culture of iPSC-3 derived hepatocytes, macrophages (both Sigma or Sigma HC3X), and endothelial cells (ETV2-Spi1), termed as HME culture in a hydrogel spheroidal and their characterisation after 17 days in culture. Representative immunohistochemistry images for the hepatocyte (NTCP, CYP3A4, CK18, MRP2, PEPCK, and AAT), cholangiocyte and non-parenchymal (CK7, CDH5, CD31, CD68, LRAT, aSMA, PDGFRb, and nestin) markers. B) Co-culture of iPSC-3 derived hepatocytes, macrophages (both Sigma or Sigma HC3X), endothelial cells (ETV2-Spi1), and hepatic stellate cells (Sigma or Sigma HC3X) termed as HMES co culture in hydrogel spheroids and their characterisation after 13 days in culture. Top raw, image on the left, shows a representative H&E staining of the HMES co-cultures. Immunohistochemistry images from HMES co-cultures for the hepatocyte (NTCP, CYP3A4, CK18, MRP2, PEPCK, and AAT) and non-parenchymal (CK7, CDH5, CD31, CD68, LRAT, aSMA, PDGFRb, and nestin) markers. The images of representatives of minimum three hydrogel spheroids all driven from two individual differentiations. Scale bar=50 μm.

### DETAILED DESCRIPTION

It is to be understood that the terminology used herein is for the purpose of describing particular embodiments only and is not intended to limit the scope of the present invention, which will be limited only by the claims.

The present invention provides a simple and fast method for producing cells in hydrogel spheroids for short- or long-term culture, especially for use in drug development, disease modelling and regenerative medicine.

In a first step of the method, cells are mixed with a self-assembling peptide to form a mixture of the cells and the peptide. In a second step of the method, an aliquot of the mixture is transferred into an aqueous solution containing salt to induce self-assembly of a hydrogel spheroid which instantly encapsulates the cells contained in the mixture within the spheroid, thereby forming a hydrogel spheroid comprising cells.

As used herein, the singular expressions “a”, “an” and “the” mean one or more. Thus, a singular noun, unless otherwise specified, carries also the meaning of the corresponding plural noun.

As used herein, the term “hydrogel” refers to a water-swollen three-dimensional (3D) polymeric network of hydrophilic polymers produced by simple reaction of one or more monomers. Accordingly, a hydrogel can retain a significant amount of water within its structure due to cross-linking of individual polymer chains, but will not dissolve in water.

As used herein, the term “self-assembling peptide hydrogel” refers to a synthetic matrix material having an ability to form a 3D hydrogel under physiological conditions by spontaneous self-assembly of the peptide component into polymer-like fibrils via non-covalent interactions and subsequent entanglement of these fibrils into an extended nanoscale polymeric network that elicits gelation of an aqueous solvent. Self-assembling peptides can be synthesized in different types with unique short or repeated amino acid sequences, some which are also commercially available from different sources including, without limitation, RADA 16-I (PuraMatrix™) by Corning Inc and KLD 12 by R&D systems. As known in the art, members of the self-assembling peptide family include, for example, RADA 16-I (RADARADARADARADA; SEQ ID NO: 1), RADA 16-II (RARADADARARADADADA; SEQ ID NO: 2), KLD12 (KLDLKLDLKLDL; SEQ ID NO: 3), KFE8 (FKFEFKFE; SEQ ID NO: 4), EAK16 (AEAEAKAKAEAEAKAK; SEQ ID NO: 5), FEFEFKFK peptide (SEQ ID NO: 6) and variants thereof.

As used herein, the term “hydrogel spheroid” refers to a self-assembled 3D peptide hydrogel scaffold having an approximately spherical shape. This term refers to the shape of the hydrogel irrespective of the presence of cells with the hydrogel.

Hydrogel spheroids as obtained by the present method have a regular homogenous shape, in contrast with the prior hydrogel drops obtained in the PuraMatrix manual of Corning by applying a hydrogel solution to the side of a well.

The term “cell spheroid” sensu strictu refers to a cell aggregate. Such cell spheroid can occur outside a matrix or within a matrix such as a hydrogel. A matrix such as a hydrogel can comprise one or more spheroids. As used herein, the term “cell spheroid of the invention” and related expressions refer to a hydrogel spheroid comprising cells that are encapsulated within and distributed throughout the spheroid. To avoid confusion with the prior art the description “hydrogel spheroid comprising cells” is used in the present application.

Such hydrogel spheroids comprising cells are obtainable by the present method of the invention.

The hydrogel spheroids comprising cells differ from conventional cell spheroids and organoids by way of their preparation method, and consequently by features of the cells in the hydrogel spheroids themselves.

In the present invention a droplet of self-assembling peptides and medium is applied to the surface of a cell culture medium via a pipette tip or other delivery device for small for volumes such as a needle of a robot used in delivering compounds to multiwell plates. The droplet is typically hanging and on outside of a pipette tip. Upon bringing the droplet, while still attached to pipette tip, into contact with a cell culture medium the droplet is released from the pipette tip sinks into the medium and forms a uniform spheroid of hydrogel.

This differs from the screening methods of PuraMatrix™, wherein a droplet rolls along the wall of a well and forms an irregular shape.

The method of the present invention further differs from prior art methods wherein a volume of self-assembling peptides and cells is flushed into medium, wherein medium is added to a volume of self-assembling peptides and cells, or wherein a preformed hydrogel of self-assembling peptides is contacted with a medium with cells.

As used herein, the term “conventional cell spheroid” refers to a simple cell aggregate formed spontaneously when adherent cells are denied an attachment surface in a hanging-drop or on an ultra-low attachment surface.

As used herein, the term “organoid” refers to a complex self-organized multicellular structure resembling a parent organ of the cells when given a scaffolding extracellular environment.

While the diameter of conventional cell spheroids is limited to a diameter of approximately 200 μm, the present hydrogel spheroids comprising cells may be as large as approximately 2.5 mm in diameter. This larger size translates, for example, into easier handling, at least partly because conventional cell spheroids are difficult to see by the naked eye. The hydrogel spheroids comprising cells are also easier to handle than organoids which are self-organized cell structures within a bulk of an ECM hydrogel matrix such as Matrigel at the bottom of a multi-well plate.

The larger size of hydrogel spheroids also provides a higher number of cells than present in conventional cell spheroids. Thus, a single hydrogel spheroid comprising cells contains, for example, more nucleic acid material (DNA or RNA), protein, and lipids that may be analysed by PCR techniques, such as qPCR and biochemical assays such as Western blot or Mass spectrometry. In addition, conventional spheroids produced in bulk in hydrogels and the organoids are heterogeneous in size and are not tuneable, while the size of hydrogel spheroids comprising cells are both homogeneous and tuneable.

In the present method, the size of the hydrogel spheroids produced will vary depending on the volume of an aliquot being used in the second method step. The higher volume, the larger the hydrogel spheroids will be, although the increase in the size/diameter is non-linear to the volume. Nevertheless, the size of the hydrogel spheroids to be produced may be adjusted by simply adjusting the volume of the aliquot to be used in the method. From a practical point of view, and in a general laboratory setting, the volume of the aliquot varies typically from 0.5 μl to 20 μl using normal (manual) pipettes common in biology laboratory. However, if the method is fully automated, the minimum volume may be as low as 0.1 μl. However, the method is not limited to the volumes exemplified above.

In some embodiments, an aliquot of 0.1 μl produces a hydrogel spheroid of about 270 μm in diameter, an aliquot of 0.5 μl produces a hydrogel spheroid of about 500 μm in diameter, an aliquot of 1 μl produces a hydrogel spheroid of about 750 μm in diameter, an aliquot of 1.5 μl produces a hydrogel spheroid of about 900 μm in diameter, an aliquot of 2 μl produces a hydrogel spheroid of about 1 mm in diameter, an aliquot of 2.5 μl produces a hydrogel spheroid of about 1.1 mm in diameter, an aliquot of 5 μl produces a hydrogel spheroid of about 1.4 mm in diameter, an aliquot of 10 μl produces a hydrogel spheroid of about 1.85 mm in diameter, an aliquot of 15 μl produces a hydrogel spheroid of about 2 mm in diameter, whereas an aliquot of 20 μl produces a hydrogel spheroid of about 2.25 mm. Thus, in some embodiments, a typical range of aliquot volumes to be used in the second step of the method varies from 0.5 μl to 20 μl, while the size of a hydrogel spheroid produced varies typically from about 500 μm to over 2 mm, more precisely to about 2.25 mm. In some embodiments, the size of a hydrogel spheroid produced may vary from about 900 μm to roughly about 2.5 mm, more precisely to about 2.25 mm.

Being basically just cell aggregates, conventional cell spheroids usually suffer from a necrotic core, at least if the diameter of the cell spheroid exceeds approximately 200 μm. Since the hydrogel spheroids comprising cells of the present invention are formed through an entirely different mechanism, necrosis in their cores is avoided by diffusion of oxygen and nutrients through the peptide hydrogel. Instead of dense cell aggregates as in conventional cell spheroids, cells comprised in the hydrogel spheroid are initially distributed evenly throughout the hydrogel. Denser cell areas may form later when the cells proliferate, connect and form a network in the spheroid hydrogel and gradually reshape their own microenvironment by complementing the provided ECM with their own secreted ECM and growth factors.

Moreover, while conventional cell spheroids lack organization, the hydrogel spheroids comprising cells of the present invention have the potential to support self-organization of complex tissue-like structures. For example, hydrogel spheroids comprising hepatic cells of the present invention exhibited a tissue like structure containing areas with biliary epithelium morphology upon extended culture, as demonstrated in Example 2.

In some embodiments, the spheroid hydrogels may be adjusted to comprise additional components such as extracellular matrix (ECM) proteins to achieve optimal cell growth, differentiation and/or self-organization owing to the presence of appropriate cell-matrix contacts. Notably, conventional cell spheroids generally and initially lack these important cell-to-ECM connections.

The above-mentioned adjusting may be achieved by coupling biologically active and functional peptides motifs onto the generic peptide or by simply adding one or more ECM proteins or peptides sequences into a mixture of cells and a self-assembling peptide hydrogel in the first step of the present method. As readily understood by those skilled in the art, the ECM proteins to be used largely depend on the preferences of the cells to be employed. For example, collagen I is particularly advantageous to be comprised in hydrogel spheroids comprising hepatic cells. Further ECM proteins to be comprised in the hydrogel spheroids comprising cells include, but are not limited to, other collagens such as collagen IV, laminins, vitronectin, fibronectin, nidogens, proteoglycans, and E-cadherin, as well as isoforms, fragments, and peptide sequences thereof. The ECM proteins and peptides may be extracted natural ECM proteins, recombinant ECM proteins or synthetic ECM proteins.

In the present method, cell density in the hydrogel spheroid to be produced may be adjusted as desired by controlling the number of cells to be mixed with a self-assembling peptide in the first step of the present method. Typically, the cell density in the mixture varies from about 1×10⁵ cells/ml to about 5×10⁷ cells/ml, preferably from about 5×10⁶ cells/ml to about 2×10⁷ cells/ml. In some embodiments, a cell density of about 1.5×10⁷ cells/ml is preferred.

The cells to be mixed with a self-assembling peptide are preferably provided in a suspension, such as a single cell suspension or a suspension comprising small cell aggregates or clumps.

Cells that are mixed with hydrogel survive during long-term of at least 8, at least 14, at least 20, at least 26 days, or at least 32 days and may proliferate and contribute to a denser structure compared to the culture at starting point. Thus the cell density after cultivation is generally as high or higher than upon formation of the hydrogel.

Typical cell densities after cultivation are accordingly about 1×10⁵ cells/ml to about 5×10⁷ cells/ml, preferably from about 5×10⁶ cells/ml to about 3×10⁷ cells/ml. or of about 2×10⁷ cells/ml.

Neither hydrogel spheroids comprising cells nor the method for the preparation thereof are limited to any particular cell type. Thus, basically any desired cell type may be employed in the method to produce the corresponding hydrogel spheroids comprising cells. For example, the cells may be primary cells of a given cell type, immortalized cell lines of a given cell type or cells derived from mesenchymal stem cells or from pluripotent stem cells.

As used herein, the term “primary cell” refers to terminally differentiated cells that can be isolated from the tissue or organ of interest. Means and methods for obtaining primary cells are readily available in the art.

As used herein, the term “mesenchymal stem cell” (MSC) refers to adult stem cells that are multipotent, i.e. can differentiate into a variety of cell types, including hepatocytes, osteoblasts, chondrocytes, myocytes and adipocytes. Means and methods for obtaining MSCs as well as differentiation thereof towards a desired cell type are readily available in the art.

As used herein, the term “pluripotent stem cell” (PSC) refers to any stem cell having the potential to differentiate into all cell types of a human or animal body, not including extra-embryonic tissues. These stem cells include both embryonic stem cells (ESCs) and induced pluripotent cells (iPSCs). Hence, the cells suitable for use in the present invention include stem cells selected from iPSCs and ESCs.

Human pluripotent stem cells (hPSCs) are preferred and they include human iPSCs (hiPSCs) and human ESCs (hESCs). ESCs, especially hESCs, are of great therapeutic interest because they are capable of indefinite proliferation in culture and are thus capable of supplying cells and tissues for replacement of failing or defective human tissue. However, use of hESCs may meet ethical challenges. According to an embodiment of the present invention, human embryonic stem cells may be used with the proviso that the method itself or any related acts do not involve destruction of human embryos.

Induced pluripotent stem cells, commonly abbreviated as iPS cells or iPSCs are a type of pluripotent stem cell artificially derived from a non-pluripotent cell, typically an adult somatic cell, by inducing a forced expression of specific genes by means and methods well known in the art. An advantage of using iPSCs is that no embryonic cells have to be used at all, so ethical concerns can be avoided. A further advantage is that production of patient-specific cells is enabled by employing iPSC technology.

Induced pluripotent stem cells are similar to natural pluripotent stem cells, such as embryonic stem cells, in many aspects. Exemplary aspects include the expression of certain stem cell genes and proteins, chromatin methylation patterns, doubling time, embryoid body formation, teratoma formation, viable chimera formation, and potency and differentiability, but the full extent of their relation to natural pluripotent stem cells is still being assessed. Induced pluripotent cells are typically made from adult skin cells, blood cells, stomach or liver, although other alternatives may be possible. Those skilled in the art are familiar with the potential of iPSCs for research and therapeutic purposes, as well as with means and methods for obtaining iPSCs. As well known in the art, pluripotency markers such POU class 5 homeobox 1 (POU5F1, OCT3/4) may be used to determine whether given cells are pluripotent. Diminished expression of pluripotency markers is indicative of cell differentiation. Stem cells retain the genotype of the individual from which they were derived, offering the opportunity to model the reproducibility of rare phenotypes in vitro, as well as to investigate and tailor treatments for patient populations with rare or idiosyncratic disease presentations. On the other hand, collections of iPSC-derived cell lines from multiple donors would facilitate studies aimed to uncover potentially significant sources of interindividual variability in drug metabolism.

For therapeutic purposes, hydrogel spheroids comprising autologous cells are preferred.

In some embodiments, hepatic cells are employed in the present method and, consequently, the hydrogel spheroids comprise hepatic cells. Non-limiting examples of hepatic cells to be used in the present invention include primary hepatocytes, hepatic cell lines and hepatocytes derived from pluripotent stem cells. Preferably, the cells are human cells.

Differentiation protocols that allow efficient differentiation of human pluripotent stem cells into hepatocyte-like cells are known in the art and include, but are not limited to, stagewise approaches where the stem cell populations are driven to definitive endoderm using substances such as Activin A, Wnt3, CHIR 99021, and/or sodium butyrate (NaB). Commercial kits for this purpose are also available. Differentiation into definitive ectoderm is followed by hepatic progenitor cell differentiation and hepatocyte maturation. Non-limiting examples of substances typically used for differentiating hepatic progenitor cells into hepatocytes include fibroblast growth factor 1 (FGF1), fibroblast growth factor 2 (FGF2), bone morphogenic protein 4 (BMP4), Dimethyl sulfoxide (DMSO), hepatocyte growth factor (HGF), oncostatin M (OSM), insulin-transferrin-selenium (ITS), and dexamethasone.

As used herein, the term “progenitor cell” is interchangeable with the term “precursor cell” and refers broadly to a descendant of a stem cell that then further differentiates to create a specialized cell type.

As demonstrated in Example 2, hepatic progenitor cells cultured within the hydrogel spheroids progressed into mature hepatocytes as evidenced by the expression of hepatic markers, and remained phenotypically stable and viable for at least 4 weeks. Hepatocytes represent the major cell population of the liver constituting up to 60-70% of the cells of the liver, the rest of the cells being known collectively as non-parenchymal cells (NPCs) including but not limited to cholangiocytes, sinusoidal endothelial cells, Kupffer cells, stellate cells and intrahepatic lymphocytes. In some embodiments, mixtures of different cell types found in the liver may be used in the present method to produce a hydrogel spheroid comprising a co-culture with hepatic cells. The presence of NPCs beside hepatocytes would be advantageous for creating a liver model that mimics the natural structure and function of the liver.

In some embodiments, cardiomyocytes are employed in the method and, consequently, the spheroid hydrogels comprise cardiac cells. Non-limiting examples of cardiac cells suitable for use in the present invention include primary cardiomyocytes, cardiomyocyte cell lines and cardiomyocytes derived from pluripotent stem cells. Means and methods for inducing cardiomyocyte differentiation of pluripotent stem cells are known in the art and include, but are not limited to, endodermal cell induced differentiation developed by Mummery et al. ((2003) Circulation, 107, 2733, Activin A and BMP4 induced differentiation developed by Laflamme et al. (2007) Nat Biotechnol 25(9), 1015, and embryoid body technique developed by Kehat et al. (2002) Circ. Res. 91, 659. Preferably, the cells are human cells.

Self-assembled peptide hydrogels, such as PuraMatrix™ made from RADA 16-I, are good supportive growth matrixes also for human neural cells. Thus, the present invention encompasses also spheroid hydrogels comprising neural cells for purposes such as neural tissue engineering, disease modelling, drug screening and neurotoxicity testing.

Non-limiting examples of further tissue types to be included in hydrogels spheroids include bone and cartilage, pancreatic islets, intestine, kidney, and lung. Accordingly, in some embodiments, one or more cell types such as osteocytes, chondrocytes, pancreatic cells, intestinal cells, renal cells, lung cells, various cancer cell types, mutated or genetically engineered cell types, or any mixture thereof may be employed in the hydrogel spheroids.

In some embodiments, mixtures of different cell types may be used in the present method to produce hydrogel spheroids comprising cell co-cultures. For example, as demonstrated in Example 3, beating cardiomyocytes were successfully co-cultured with hepatic progenitor cells in the same hydrogel spheroid in hepatic maturation medium such that the cardiomyocytes retained their phenotype and beating activity for several weeks. This co-culture setup provides new opportunities for studying multiple tissue types in one system, a feature that is particularly valuable for organ-on-chip projects or drug metabolism and toxicity studies.

The mechanical strength of the self-assembled hydrogel spheroid to be produced may be easily adjusted by controlling the amount of a self-assembling peptide to be used in a first step of the present method. For example, when PuraMatrix™ is employed, concentration of 0.1% w/v creates a soft hydrogel that exhibits a relatively weak mechanical strength, whereas concentration of 1% w/v creates a hydrogel with greater mechanical strength. Usually, the amount of the self-assembling peptide to be used varies between these concentration ranges. The mechanical strength could be also adjusted by addition of one or more other matrix proteins, such as Collagen Type I, to the self-assembled hydrogel.

To avoid polymerization of the self-assembling peptide to be used too early, already during the first step of the present method, cells to be mixed with the peptide are typically in a solution that does not contain any salts. However, to maintain viability of the cells, the medium should be isotonic to the cells. Thus, isotonic sugar solutions are preferably solutions in which the cells to be mixed with the self-assembling peptide should be provided. Non-limiting examples of such media include sucrose and/or glucose solutions, such as those containing about 10-20% of sucrose and/or glucose.

In the second step of the present method, hydrogel spheroids encapsulating the cells are formed instantly when an aliquot of a mixture comprising the cells and the self-assembling peptide is transferred into an aqueous salt solution. This is caused by an immediate gelation due to a reaction of the self-assembling peptide and the salt present in the solution, resulting in a self-assembled peptide hydrogel.

Different types of aqueous salt solutions may be used in the second step of the method, depending on the ultimate application area of the produced hydrogel spheroids comprising cells. If these hydrogels spheroids comprising cells are intended for therapeutic purposes, it may be beneficial to use aqueous salt solutions other than cell culture media which often contain ingredients such as colouring agents. Non-limiting examples of such salt solutions include physiological saline solutions, such as isotonic normal saline or Ringer's solution and modifications thereof.

In some embodiments, cell culture medium may be used as the aqueous salt solution. As used herein, the term “basal medium” refers to a cell culture medium composed of components essential for cell growth and maintenance including amino acids, glucose and salt ions such as calcium, magnesium, potassium, sodium and phosphate, as is well known in the art. Non-limiting examples of commercially available basal media suitable for use in the present method include KnockOut Dulbecco's Modified Eagle's Medium (KO-DMEM), Dulbecco's Modified Eagle's Medium (DMEM), Minimal Essential Medium (MEM), Basal Medium Eagle (BME), RPMI 1640, F-10, F-12, William's E medium, Glasgow's Minimal Essential Medium (G-MEM), Iscove's Modified Dulbecco's Medium and any combinations thereof. Those skilled in the art can easily selected an appropriate basal medium to be used in the present method, depending on different variants such as the cell type to be employed in the produced hydrogel spheroids comprising cells.

In some embodiments, the basal medium may be supplemented with ingredients used in practically every cell culture medium including antibiotics, L-glutamine, essential and non-essential amino acids, fatty acids, and serum, serum albumin or a serum replacement, preferably a defined serum replacement.

To achieve optimal cell growth and differentiation, the basal medium may be supplemented with appropriate bioactive molecules such as growth factors and ECM proteins, and/or other molecules. These additional supplements depend on the cells to be employed in the hydrogel spheroids comprising cells. For example, if stem cells or progenitor cells are to be differentiated, the cell culture medium should contain appropriate differentiation-inducing agents. Those skilled in the art can easily selected such agents depending on the lineage towards which the stem cells or progenitor cells are to be differentiated.

Preferably, the cell culture medium to be used is xeno-free. As used herein the term “xeno-free” refers to absence of any foreign material or components. Thus, in case of human cell culture, this refers to conditions free from non-human animal components. In other words, when xeno-free conditions are desired, for example, for the production of hydrogel spheroids comprising cells for human therapy, all components of any cell culture media must be of human or recombinant origin.

Traditionally, serum, especially foetal bovine serum (FBS), has been used in cell cultures to provide essential growth and survival components for in vitro cell culture of eukaryotic cells. It is produced from blood collected at commercial slaughterhouses from cattle bred to supply meat destined for human consumption. “Serum free” indicates that the culture medium contains no serum, either animal or human.

Preferably, the cell culture medium to be used is defined. Undefined media may be subject to considerable dissimilarities due to natural variation in biology. Thus, undefined components in a cell culture may compromise the repeatability of cell model experiments e.g. in drug discovery and toxicology studies. Hence, “defined medium” or “defined culture medium” refers to a composition, wherein the medium has known quantities of all ingredients.

To obtain a defined cell culture medium, serum that would normally be added to the medium for cell culture is replaced by known quantities of serum components, such as albumin, insulin, transferrin and possibly specific growth factors (e.g., basic fibroblast growth factor, transforming growth factor or platelet-derived growth factor).

However, in some embodiments, a chemically defined medium is preferred. As used herein, the term “chemically defined medium” refers to a growth medium in which all of the chemical components are known. A chemically defined medium is entirely free of animal-derived components and represents the purest and most consistent cell culture environment. By definition, chemically defined media cannot contain foetal bovine serum, bovine serum albumin or human serum albumin as these products are derived from bovine or human sources and contain complex mixes of albumins and lipids. Thus, chemically defined media differ from serum-free media in that bovine serum albumin (BSA) or human serum albumin (HSA) is replaced with either a chemically defined recombinant version (which lacks the albumin associated lipids) or a synthetic chemical, such as the polymer polyvinyl alcohol, which can reproduce some of the functions of BSA/HSA. Commercially available serum replacement formulations include, but are not limited to, KnockOut™ Serum Replacement (Ko-SR) and its xeno-free version KnockOut™ SR XenoFree CTS™, both commercially available from Life Technologies.

The hydrogel spheroids comprising cells may be used for various medicinal purposes such as drug development (including different aspects and assays such as drug efficacy and/or toxicity screenings, investigative/mechanistic toxicology studies, target discovery/identification, drug repositioning studies, pharmacokinetics and pharmacodynamics assays), disease modelling and regenerative medicine.

In drug development, hydrogel spheroids comprising cells can be used for screening of drugs and other substances such as small molecule drugs, peptides, antibodies, nanobodies, affibodies, aptamers and polynucleotides, for their effects of the cell spheroids. In some embodiments, the aim of the screening may be to identify candidate compounds for the presence or absence of a pharmacological effect on a cell type, or a mixture of cell types, comprised in the hydrogel spheroids. The presence or absence of a pharmacological effect may be determined based on various readouts including, but not limited to, a change in viability, proliferation rate, morphology, migration, cellular stress response, secretion of proteins and cytokines, lipoproteins and lipids as well as extracellular matrix components, gene and protein synthesis and marker expression, DNA synthesis or metabolic activity, beating and/or electrophysiological properties, calcium flux, energy consumption and mitochondrial function, as compared to corresponding effects in control hydrogel spheroids comprising cells, such as hydrogel spheroids comprising cells contacted with a control compound or hydrogel spheroids comprising cells not contacted with any test compound.

As readily understood by those skilled in the art, the readout used in the assay depends on the pharmacological effect whose presence or absence is to be determined. Moreover, the pharmacological effect to be determining, and hence the readout to be used, largely depends on the cell type and disease in question. The pharmacological effect to be determined may be a desired pharmacological effect or an adverse effect. Thus, the assay may be used in screening for desired pharmacological effects or in screening for adverse pharmacological effects or any side effects.

The above aspect of the invention may be expressed in different ways. In some embodiments, the invention provides an assay for identifying a candidate compound for drug development, the assay comprising the steps of:
i. contacting the hydrogel spheroid comprising cells with a test compound,
ii. detecting whether the test compound has a pharmacological effect on the cells in the hydrogel spheroid, and
iii. identifying the test compound as a candidate compound for drug development if the detected pharmacological effect is the desired pharmacological effect.

In some embodiments, the invention provides an assay for identifying a candidate drug for the treatment of a disease. Such an assay may be formulated as an assay comprising the following steps:
i. contacting the hydrogel spheroid comprising cells showing a disease phenotype of interest with a test compound,
ii. detecting whether the test compound has an effect on the cells in the hydrogel spheroid, and
iii. identifying the test compound as a candidate drug for the treatment of the disease, if the detected effect is a positive pharmacological effect associated with amelioration of the disease.

In case of hydrogel spheroids comprising hepatic cells, obtaining a readout of a pharmacological effect may involve, for example, carrying out viability assays (e.g. cancer) using e.g. CellTiter-Glow™ kit (provided by Promega) and ATP production measurements, determining the expression of gene and protein of interest, determining the synthesis and secretion of glycogen serving as an indication of gluconeogenesis, or determining synthesis of urea, plasma proteins or relevant cytokines, determining necrotic cell death by measuring the release of cytoplasmic enzymes e.g. lactate dehydrogenase (LDH) leaked from cell membrane, or measuring the activity of phase I and phase II drug metabolizing enzymes such as CYP1A2, CYP2D6, CYP2E1, CYP2C9, CYP3A4, and UDPG.

In case of hydrogel spheroids comprising cardiac cells, obtaining a readout of a pharmacological effect may involve, for example, measuring field potential by a micro electrode array (MEA), measuring contractile force and electrical conductivity, measuring LDH for evaluating necrotic cell death, measuring calcium transients, performing cell beating analysis by available sophisticated imaging or video analysis software or carrying out quantitative gene expression assays.

In case of hydrogel spheroids comprising neural cells, obtaining a readout of a pharmacological effect may involve, for example, carrying out cell proliferation or viability assays, analysing calcium flux or performing calcium imaging, measuring field potential by a micro electrode array (MEA), or carrying out quantitative gene expression assays.

It has been shown herein that co-culturing hepatocytes and cardiomyocytes in a single hydrogel spheroid in a single system (hydrogel and culture media) is possible. Alternatively, the cells in the hydrogel spheroids could be derived from single tissue types individually and then be connected to each other on a single system. For instance, hydrogel spheroids comprising hepatocytes and cardiomyocytes could be generated individually and then be connected via a microfluidic system or multi-organ- or human-on-a-chip platform recapitulating primary aspects of the in vivo crosstalk between liver and heart enabling pharmacological studies in a more accurate manner in a single system to e.g. predict adverse effects in preclinical studies or chronic disease modelling. Nevertheless, this system is not limited to hepatocytes and cardiomyocytes only. Thus, other tissue types could be also employed in the platform. Testing two or more compounds simultaneously or sequentially enables determination of any interaction effects of the compounds.

In toxicity studies, test compounds are screened for potential toxic effects. Non-limiting examples of toxic effects include cytotoxicity that can be determined e.g. on the basis of cell morphology, viability, apoptosis, membrane integrity, mitochondrial dysfunction, and oxidative stress; hepatotoxicity that can be determined e.g. on the basis of loss of ability of producing and/or secreting serum proteins such as albumin and urea, or the maintenance of hepatic cytochrome P450 activity showing their metabolic competent and inducibility; cardiotoxicity that can be determined e.g. on the basis of electrical activity measurements using e.g. MEA or force measurements using an optical detection system and/or video analysis platform and calcium flux measurement; and neurotoxicity that can be determined e.g. on the basis of calcium flux measurements, or multielectrode arrays (MEA).

In some embodiments, an assay for determining toxicity of a test compound may be formulated as an assay comprising the steps of:
i. contacting the hydrogel spheroid comprising cells with the test compound,
ii. assessing whether the test compound has an effect on the cells in the hydrogel spheroid, and
iii. determining the toxicity of the test compound based on the assessed effect.

The present hydrogel spheroids comprising cells may also be used for disease modelling. For instance, iPSC lines generated from patients with genetic disorders (e.g. Tangier disease, alpha 1 antitrypsin (A1AT) deficiency, Long QT syndrome) could be used to create hydrogels spheroid comprising cells, which have the disease phenotype of interest. Another option is to use engineered cell lines created by genome editing techniques (e.g. CRISPR/Cas9) showing the phenotype of disease of interest (E.g. alpha 1 antitrypsin deficiency, mutation in LDL receptor (LDLR), Glycogen storage deficiency (type 1a and 1b), or rare diseases such as mitochondrial DNA depletion syndrome type 3 (MTDPS3)). Creating a diseased model can be also achieved by infecting the cells with an external agent like viruses or parasites (e.g. hepatitis type C or Plasmodium falciparum (cause of Malaria)). A further way of creating a disease model is to cause an artificial insult/injury by treating the cells by harmful or stressful agents and creating a model showing the phenotype of the disease of interest. For instance, treating the hepatocytes with high doses of fatty acids would lead to accumulation of fats in hepatocytes causing liver damage and fibrosis similar to what is observed in the liver of the patients with non-alcoholic fatty liver disease (NAFLD). A still further way of creating diseased models would be to use cell lines created from a tumour biopsy. Notably, the above examples are illustrative only, and do not limit the present invention.

All the above-mentioned cell models provide valuable means for studying disease mechanisms and interactions between different cell/tissue types e.g. in metabolic diseases and/or oncology, and facilitating drug discovery and safety.

The hydrogel spheroids comprising cells may also be used for therapeutic applications, for example to restore normal organ function in a subject in need thereof. In this regard, it is noted that synthetic self-assembly peptide hydrogels are biocompatible, i.e. material that, upon administration in vivo, is compatible with living tissues and does not induce substantial undesirable long-term effects, such as toxicity reactions or immune responses. Besides, iPSC-derived tissues have been already opened their way into clinical use. It is thus envisaged that the hydrogel spheroids comprising cells may be transplanted in vivo for providing critical support for the host tissue in the patients e.g. with compromised liver function as a parallel or even substitute of bioartificial liver devices of liver transplantation.

A further aspect of the invention relates to a kit comprising one or more substances needed for producing a hydrogel spheroid comprising cells. Thus, the kit may comprise a self-assembling peptide composition, optionally tailored depending on the one or more cell types to be encapsulated within the peptide hydrogel spheroid and/or tissue type to be mimicked. The self-assembling peptide composition to be provided in the kit is not limited to those comprising any of the self-assembling peptides exemplified above. In some embodiments, the kit may optionally comprise additional peptides and/or proteins complementing the selected self-assembling peptide to create a more favourable environment for growth and differentiation of the cell type of interest. The kit may also comprise cells (in either live or frozen format) to be encapsulated within the hydrogel spheroid, preferably as frozen cells. The kit may also comprise frozen hydrogel comprising cells. Optimized isotonic non-salt solutions such as sucrose and/or glucose solutions and/or enzymes required for cellular detachment prior to hydrogel formation may also be provided in the kit. In some embodiments, the kit may also comprise an optimized medium including essential growth factors for differentiation of the cells in the hydrogel spheroids towards cell or tissue types of interest and/or optimized medium including essential growth factors for a long-term culture and maintenance of the cells in the hydrogel spheroids.

In accordance with the above, in some embodiments, the kit comprises:
a) a self-assembling peptide composition, optionally tailored depending on the one or more cell types already encapsulated or to be encapsulated within the peptide hydrogel spheroid and/or tissue type to be mimicked, and
b) one or more components selected from the following:
   i) additional peptides and/or proteins, such as one or more ECM proteins or peptides, complementing the selected self-assembling peptide to create a more favourable environment for growth and differentiation of the cell type of interest,
   ii) an aqueous salt-containing solution to induce self-assembly of a hydrogel spheroid,
   iii) cells to be encapsulated with the hydrogel spheroid, preferably as frozen cells
   iv) Cells already encapsulated within the hydrogel spheroid, preferably as frozen hydrogel comprising cells
   v) an optimized isotonic non-salt solution such as a sucrose and/or glucose solution,
   vi) one or more enzymes required for cellular detachment,
   vii) optimized medium including essential growth factors for differentiation of the cells in the hydrogel spheroids towards cell or tissue types of interest, and
   viii) optimized medium including essential growth factors for a long-term culture and maintenance of the cells in the hydrogel spheroids.

In some embodiments, the hydrogel spheroids comprising cells can be mass produced in house and then delivered to the end-users as frozen or ready-to-use plates or tubes.

### EXAMPLES

### Example 1. Preparation of Hydrogel Spheroids Comprising Hepatic CELLS Human Induced Pluripotent Stem Cells (iPSCs)

Human iPSCs expressing characteristic pluripotency markers were maintained as described earlier [Kiamehr et al. (2017) Dis Model Mech 10(9), 1141-1153; Kiamehr et al. (2019) J Cell Physiol 234(4), 3744-3761]. In brief, iPSCs were maintained at 37° C. in 5% CO₂ on mouse embryonic fibroblasts (MEFs, Applied StemCell, Cat. No. ASF-1223) in Knock-out Dulbecco's Modified Eagle Medium (KO-DMEM) supplemented with 20% KnockOut Serum Replacement (KO-SR), 2 mM Glutamax, 0.1 mM 2-mercaptoethanol (2-ME) (all from Gibco®), 1% nonessential amino acids (NEAA) and 50 U/ml penicillin/streptomycin (both from LONZA). The medium was supplemented with 4 ng/ml human basic fibroblast growth factor (bFGF, R&D system). For hepatic differentiation, iPSCs were transferred to feeder free cell culture on Geltrex (1:100) in mTeSR1 medium.

Hepatic Differentiation and Generation of Hydrogel Spheroids Comprising iPSC-Hepatocytes

The 2D hepatic differentiation applied here was based on the differentiation methods disclosed earlier [Kiamehr et al. (2019) J Cell Physiol 234(4), 3744-3761].

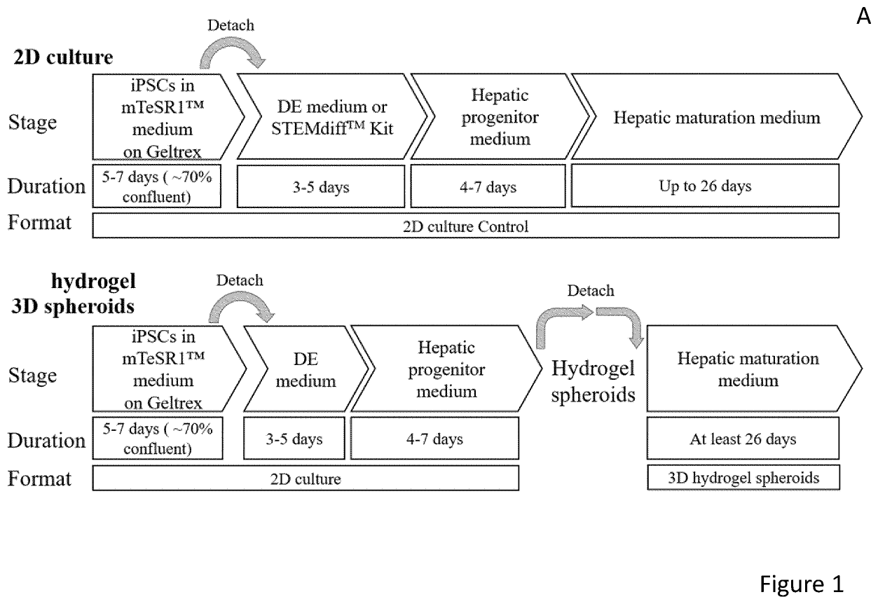

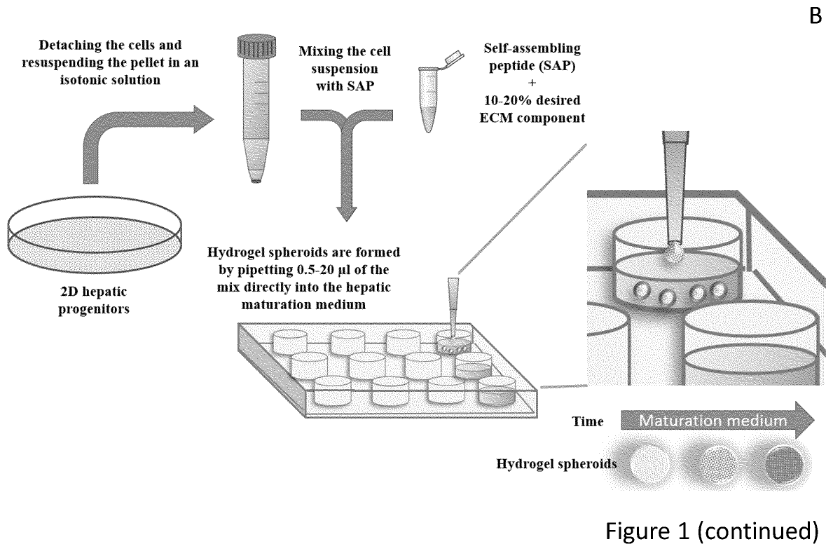

iPSC-1: After reaching 70% confluency, iPSCs were detached by Versene (Gibco) and resuspended in mTeSR1 supplemented in 10 μM Rock inhibitor and cultured on Geltrex (1:50) for 24 hours. Cells were then differentiated to definitive endoderm (DE) using a commercial kit (STEMdiff™, Cat No. 05111). The differentiation in DE stage was performed according to the kit manufacturer's instructions. The hepatic progenitor differentiation was initiated by switching the medium to KO-DMEM+20% KO-SR, 1 mM Glutamax, 1% NEAA, 0.1% β-ME, and 1% DMSO for 6-7 days. Medium was then switched to maturation medium consisting of 2-hydroxy-4-methoxybenzophenone (HBM; cc-3199, Lonza) supplemented with SingleQuots™ complemented with 25 ng/ml hepatocyte growth factor (HGF; PHG0254, life technologies) and 20 ng/ml oncostatin M (OSM, 295-OM, R&D systems). Cells were kept in the maturation medium up to 26 days. Medium was changed every other day (FIG. 1A).

iPSC-1 & 2: After reaching 70% confluency, iPSCs were detached by Versene (Gibco), centrifuged on 200 g, and re-suspended in DE medium: RPMI+Glutamax supplemented with 1×B27, 100 ng/ml Activin A, 50 ng/ml Wnt3, and 10 μM Rock inhibitor. The cell suspension was seeded with 5-10×10⁴/cm² density. Next day Rock inhibitor was replaced with 0.5 μM sodium butyrate (NaB) until day 3-5 of differentiation. The hepatic progenitor and maturation stages were similar to the method used for iPSC-1 (FIG. 1A).

Hydrogel spheroids: Hepatic progenitors were washed twice by DPBS and detached using either Gentle Cell Dissociation buffer (STEMCELL Technologies, Cat: 07174, Canada) for 25 minutes or by Tryple (Gibco™, Cat: 12563011) for 5-7 minutes, detached in HCM, transferred to 15 ml falcon tube, centrifuged on 200 g for 4 minutes, resuspended in 10% sucrose and centrifuged again at 200 g for 4 minutes, and at last, cell pellets were resuspended in 10% sucrose. Cell suspension (1.5-3×10⁷/ml) was mixed (1:1) with a self-assembling peptide (SAP) PuraMatrix™ (Corning®, Ref: 354250, MA, USA)+20% Bovine Collagen I (Gibco, Ref: A10644-01, Auckland, NZ). Hydrogel spheroids were immediately made by pipetting 2-10 μl of the mixed cells and hydrogel directly in the maturation medium. Hydrogel spheroids were formed instantly while sinking in the medium as a result of the immediate gelation due to the reaction of the SAP and the salt presented in the medium (FIG. 1B). The medium was changed after three times within one hour from generation of the hydrogel spheroids. Hydrogel spheroids were cultured in the maturation medium for up to 26 days in a static condition and were maintained at 37° C. in 5% CO₂. Medium was changed every other day.

Hydrogel Spheroids Comprising HepG2 Cells

Hydrogels spheroids comprising HepG2 cells were generates with the same method used for iPSC-hepatic cells. In brief, HepG2 cells were collected from 2D culture using Trypsin-EDTA and hydrogel spheroids were generated using a mix of cells (3×10⁷/ml) SAP+20% Collagen I into the RPMI medium+10% FBS.

### Example 2. Characterization of Hydrogel Spheroids Comprising Hepatic Cells Morphology and Viability

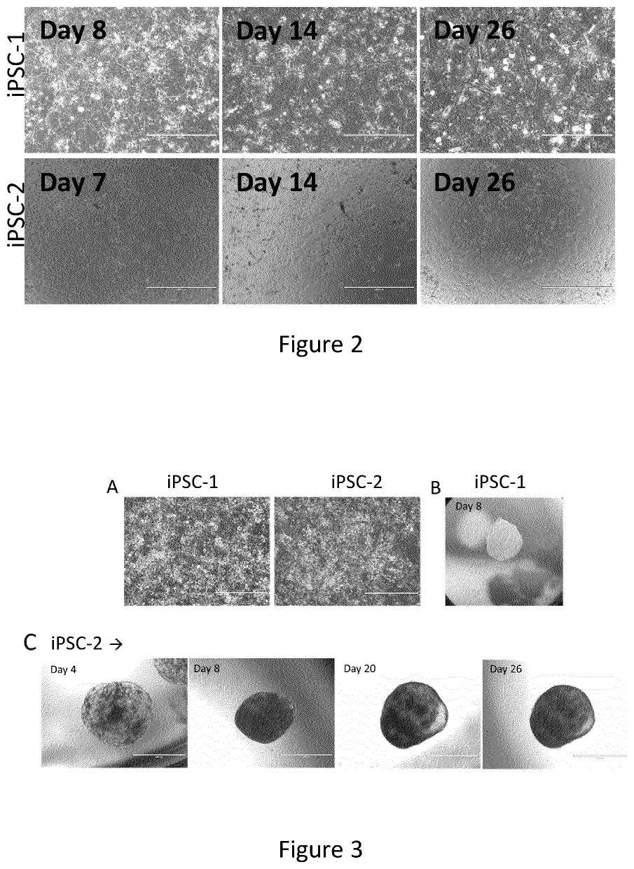

In 2D culture, cells differentiated from iPSC-1 and iPSC-2 reached the typical morphology of matured hepatocytes with polygonal shapes after a few days of culture in maturation medium and kept their morphology for about a week, after which they began to gradually lose morphology. Hepatocytes derived from iPSC-1 remained viable during the 26 days of the culture in the maturation medium, however hepatocytes differentiated from iPSC-2 started to die and detach from the culture surface already in the second week of the culture, and on the 4^{th} week, they had significantly lost their morphology and viability (FIG. 2).

2D hepatic progenitor cells (FIG. 3A) were detached and hydrogel spheroids were generated using a mix of cells, PuraMatrix™, and Collagen I, and pipetting the mix directly into the maturation medium (FIG. 2B). Hepatic cells within the hydrogel spheroids started to connect and migrate to reform their environment within the hydrogel. After a week in the culture, the entire area of the hydrogel spheroids was occupied by the hepatic cells (FIGS. 3 B&C).

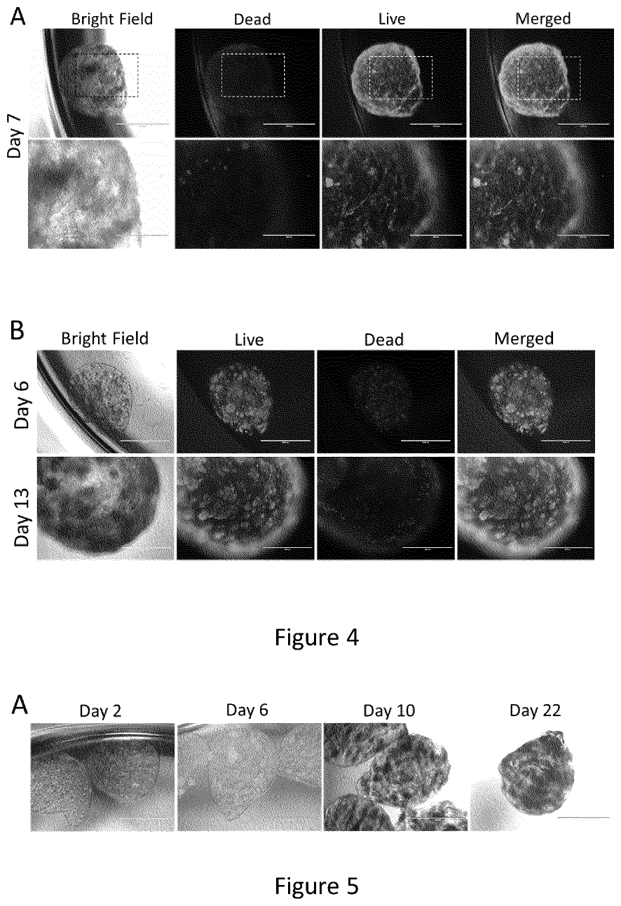

Live/dead assay was performed using a mix of 0.2 μM fluorescent calcein-AM staining live cells (green colour), and 1 μM ethidium homodimer-1 staining dead cells (red colour). After 30 minutes of treatment cells in the hydrogel spheroids were imaged with an Evos FL cell imaging microscope. Results showed that hepatocytes remained viable during the culture and number of dead cells within the hydrogel spheroids were minimal (FIGS. 4A and 4B).

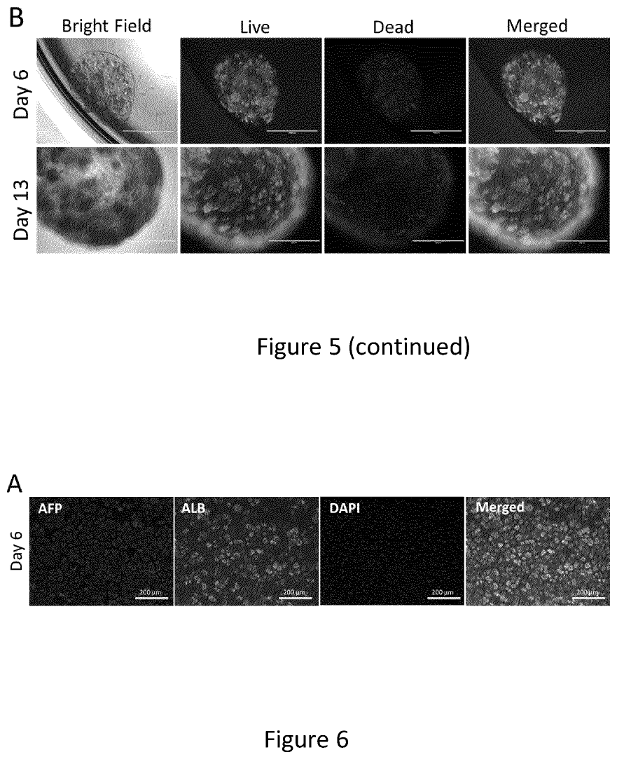

Also HepG2 cells were able to grow well in the hydrogel spheroids. Initially, the cells formed small clusters within the structure but gradually spread such that, after two weeks, they were densely occupying the entire areas of the hydrogel spheroids and particularly the peripheral area of the hydrogel spheroids (FIG. 5A). After about 3 weeks, HepG2 started to outgrow from the hydrogel spheroid surface. Live/dead assay showed that HepG2 cells in the hydrogel spheroids remained mostly viable during the entire culture (FIG. 5B).

Immunocytochemistry

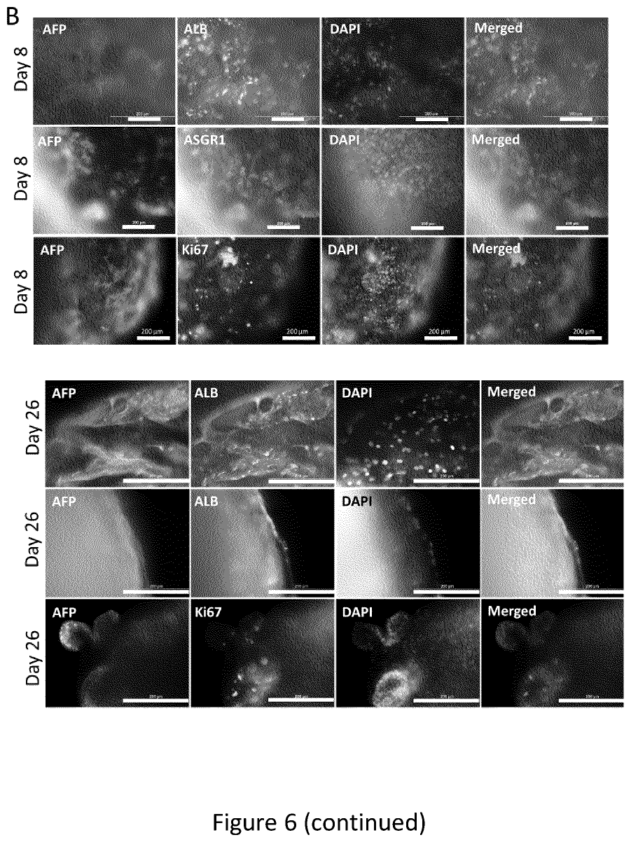

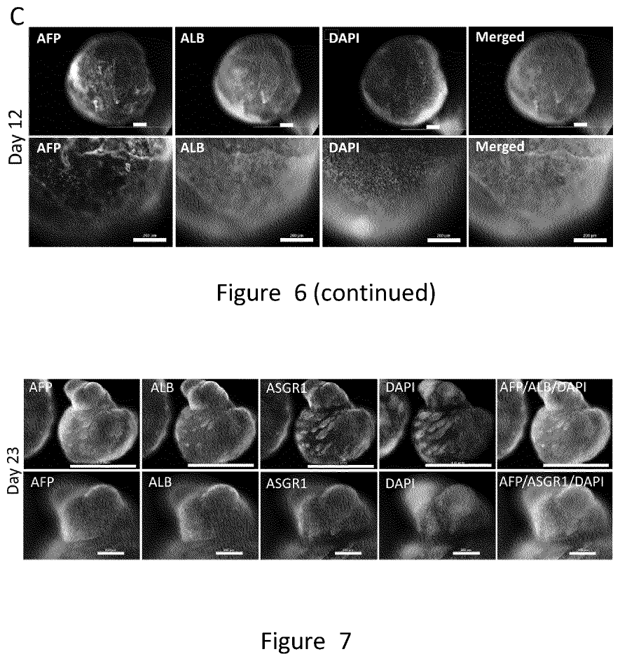

Immunostaining showed that hepatic progenitor cells could maintain their phenotype in the hydrogel spheroids and become mature as was confirmed by two hepatic markers of AFP (immature marker) and ALB (mature marker), like what was observed already in 2D culture (FIGS. 6A and 6B). The expression of AFP and ALB in protein levels was shown at time points day 8 and 26 for the hepatic cells in the hydrogel spheroids derived from iPSC-1 (FIG. 6B) and day 12 for the hepatic cells in the hydrogel spheroids derived from iPSC-2 (FIG. 6C). ASGR1, another marker for mature hepatocytes, was also expressed in hepatic cells in hydrogel spheroids (FIG. 6B). Within the hydrogel spheroids, Ki67 positive cells were detected at both days 8 and 26 (FIG. 6B) indicating the presence of dividing cells in the hydrogel spheroid structure. On day 26, Ki67 positive cells were mainly localized in denser areas compared to day 8, which were mostly scattered throughout the hydrogel spheroids. This data shows that hepatic progenitor cells could become mature in the hydrogel spheroids and keep their phenotype during the entire 4 weeks of the culture. Additionally, it shows that dividing cells could grow and contribute to create a denser environment in the hydrogel spheroids.

HepG2 cells remained their phenotype during the entire length of culture and expressing AFP, ALB, as well as ASGR1, confirmed by immunocytochemistry (FIG. 7).

Histology

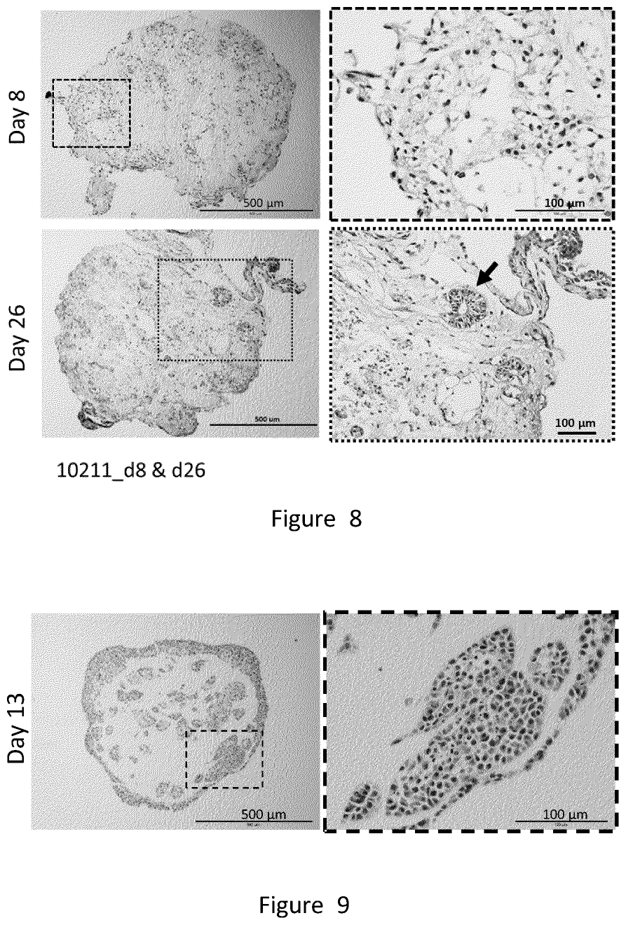

Haematoxylin and Eosin (H&E) staining showed the distribution of hepatic cells throughout the hydrogel spheroids. On day 26, the density of the cells in the hydrogel was higher compared to the hydrogel spheroids on day 8, confirming our previous observation from both morphology and immunostaining (FIG. 8). As a result, cells in the hydrogel spheroids on day 26 showed a more tissue like structure, containing also cells/areas with biliary epithelium morphology (FIG. 8, lower right panel, black arrow) indicating the existence of other hepatic lineage cell types in the structure mimicking the native liver tissue, where cells were highly in contact with each other with divers but relevant cell types creating and reshaping physiologically relevant microenvironment.

In hydrogel spheroids comprising HepG2 cells, cells were growing densely, distributed throughout the hydrogel as was shown by H&E staining (FIG. 9), however majority of cells were localized at the periphery of the hydrogel spheroids with some sparse dense areas at the core of the hydrogel spheroids. A higher magnification view showed (FIG. 9, right image) the alignment of HepG2 cells both on the surface and inside the periphery area creating an environment with maximized cell-cell contact.

Immunohistochemistry

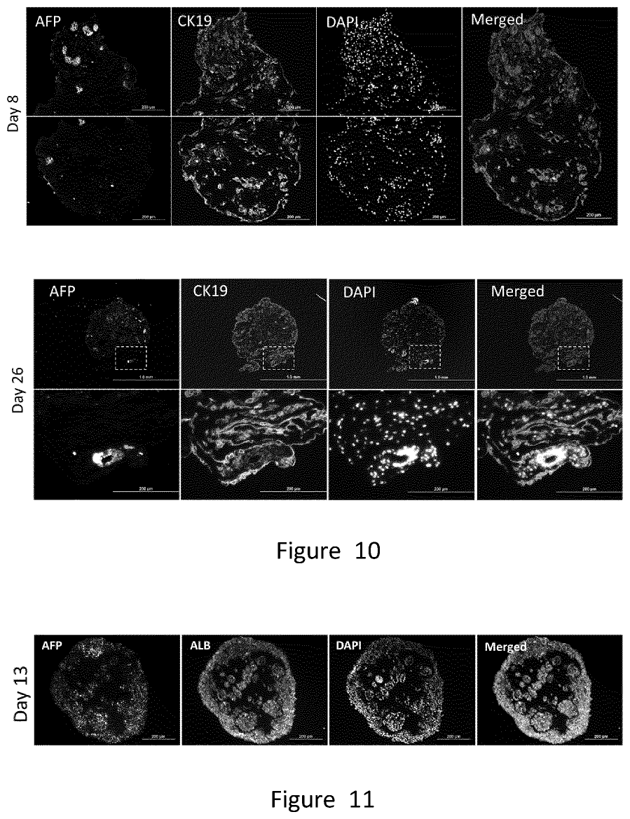

Immunohistochemistry of the hydrogel spheroids comprising iPSC-hepatic showed the presence of AFP and CK19 (cholangiocyte marker) positive cells within the hydrogel spheroids. This confirmed our previous observation from H&E data that beside hepatocytes, cholangiocytes (CK19 positive cells) were also differentiated from the hepatic progenitors. Interestingly AFP positive areas, were mostly localized in the denser areas inside the hydrogel spheroid structure (FIG. 10).

HepG2 cells were also shown to keep their morphology by expressing hepatic markers of AFP and ALB (FIG. 11).

Gene Expression by gPCR

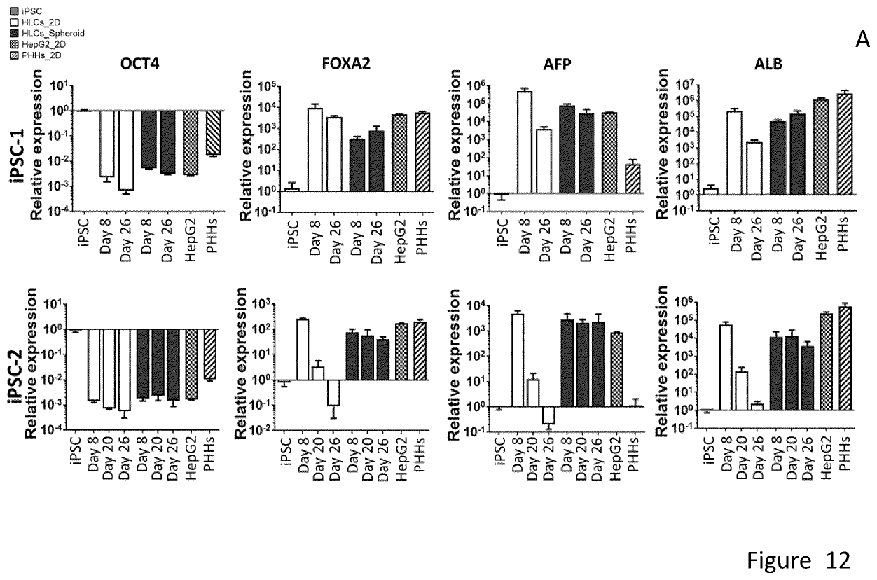

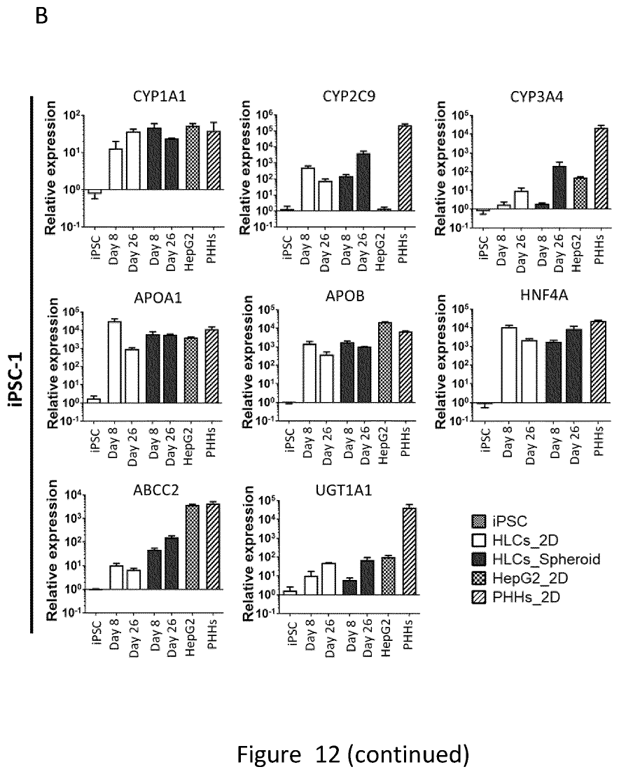

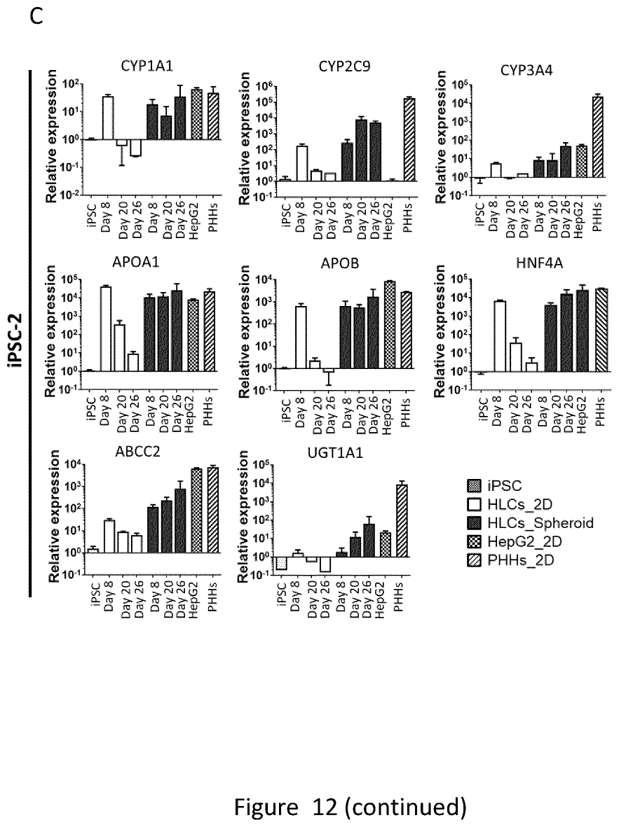

Using qPCR, the expression level of some of the key genes important in hepatic maturity and functionality was studied in hydrogel spheroids comprising hepatic cells, and the expression levels were compared with their 2D hepatic counterparts as well as with two standard hepatic cell types: primary human hepatocyte (PHHs) and HepG2 cells (FIGS. 12A and 12B). The results showed that the expression of all studied genes in hepatic cells in hydrogel spheroids either remained relatively constant or upregulated during the culture with a peak on 26 days. This is while in their counterpart 2D culture the expression of most those genes were significantly downregulated. OCT4, pluripotency marker, remained downregulated in all groups. This proves that hydrogel spheroids comprising hepatic cells are suitable for long-term cultures and have addressed the limitation faced when working with 2D cultures. In addition, the expression of most key genes shown to be higher in a hydrogel spheroid compared to the 2D cultures indicating superiority of the hydrogel spheroids for the growth of hepatic cells.

### Example 3. Co-Culture of Beating Cardiomyocytes with Hepatocytes in Hydrogel Spheroids

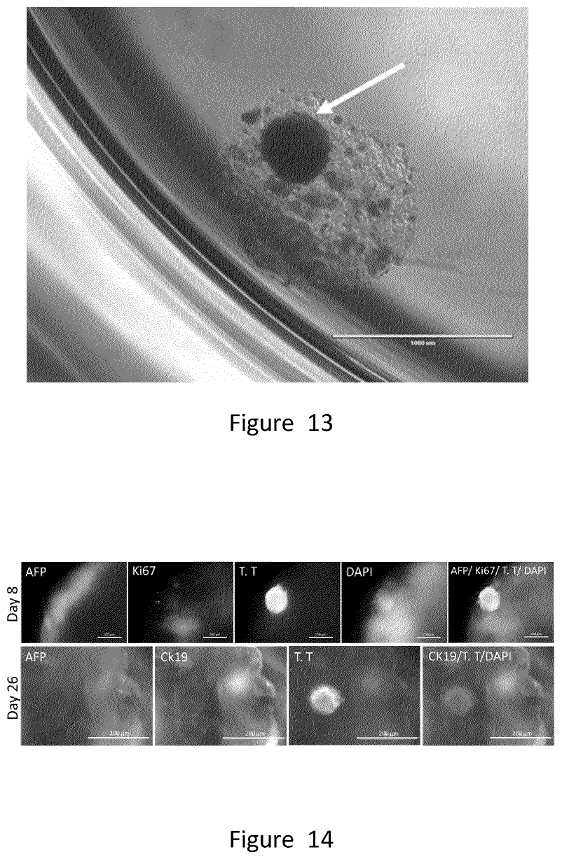

In a separate attempt, beating cardiomyocyte aggregates were co-cultured with hepatic progenitor cells in the same hydrogel spheroid and cultured in hepatic maturation medium (FIG. 13). Cardiomyocytes could keep their phenotype and their beating for several weeks in this novel setup. It is shown here, that a) it is possible to co-culture hepatic and cardiac tissues in the same hydrogel spheroid, b) beating cardiomyocytes remained their phenotype and their beating in the hepatic maturation medium. This provides the opportunity of studying both tissue types in one system/medium particularly valuable from the aspect of organ-on-chip projects or drug metabolism and toxicity studies.

### Example 4. Hepatic Differentiation Derived from iPSC-3

2D culture: A hiPSC line (Sigma 0028, Sigma-Aldrich) was genetically engineered to inducibly overexpress HNF1A, FOXA3 and PROXI (named as Sigma HC3X, or iPSC-3) as described earlier in the art [Boon et. al. (2020) Nat. Commun. 11(1), 1393]. Single hiPSCs were cultured on Matrigel-coated plates in mTeSR medium (Stem Cell Technologies). When cells reached 70-80% confluency, the differentiation was started using a sequences of cytokine cocktails (all from Peprotech) in liver differentiation medium (LDM) until day 12; after which, 15 ml of MEM Non-Essential Amino Acids Solution and 7.5 ml of MEM Amino Acids Solution (Thermo Scientific) per 100 ml of LDM was added to the culture medium for 2 days accompanied with 2% DMSO, and from day 14 onward, glycine at a concentration of 20 g/L was added combined with the amino acids until the end of the culture as described in the art [Boon et al. (2020) cited above].

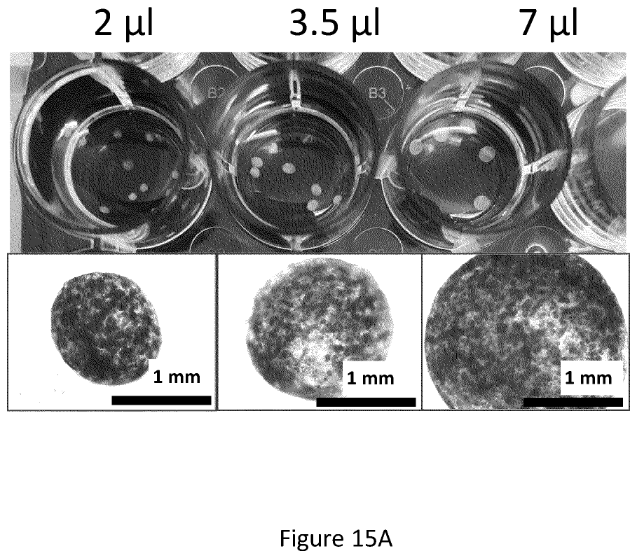

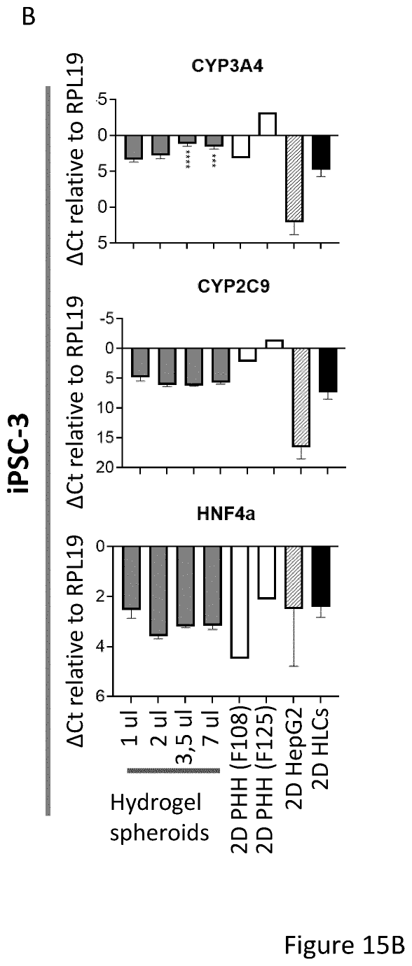

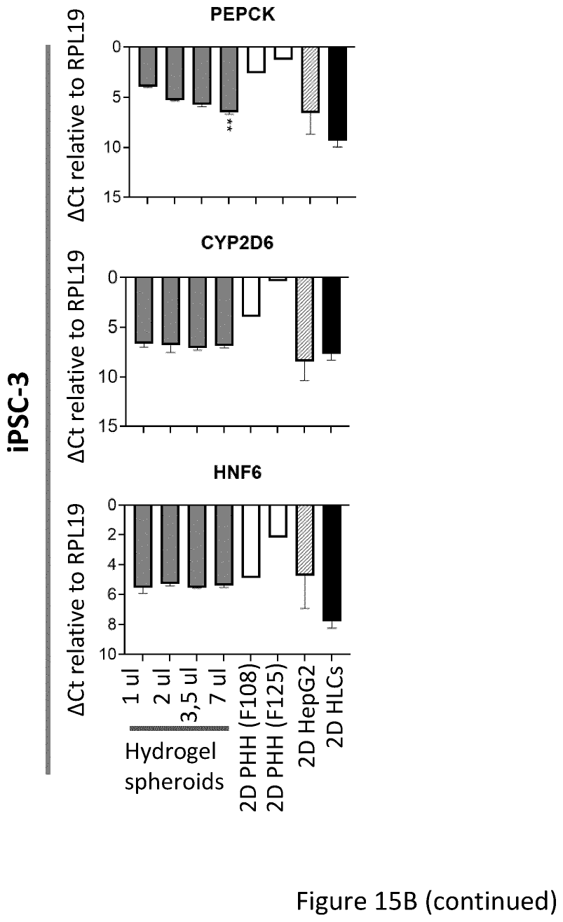

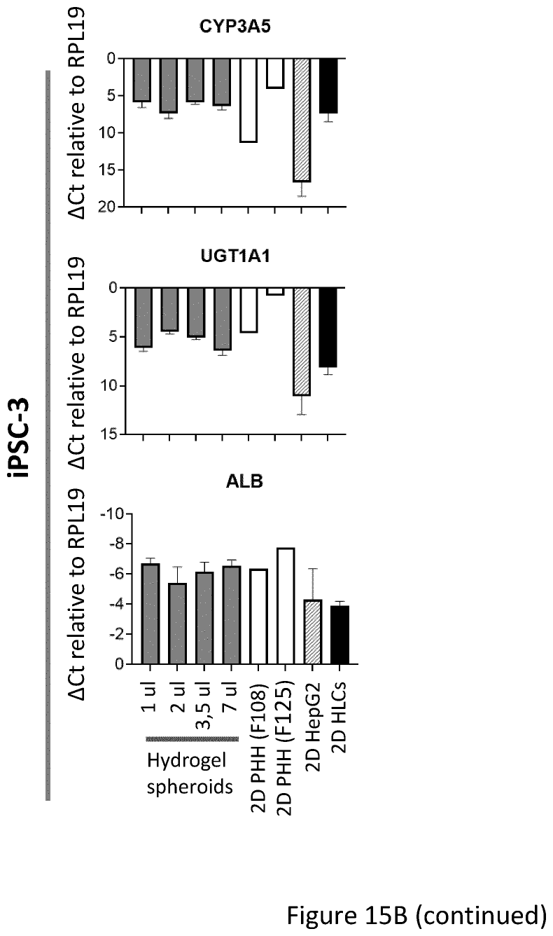

3D culture: in order to generate the hydrogel spheroids, hepatic progenitors were collected as single cells as described in example 1 and transferred to the self-assembling peptides (PuraMatrix™) supplemented with 20% Bovine Collagen I (Gibco, Ref: A10644-01, Auckland, NZ). In order to generate hydrogel spheroids in various sizes, 1, 2, 3.5, and 7 μl of the hydrogel mixed with cells were pipetted directly on the surface of the hepatic medium. Hydrogel spheroids were formed in various sizes (depending on the applied volume) and were maintained in hepatic medium for 32 days (FIG. 15 A).

Gene Expression by qPCR

RNA extraction using Qiazol lysis buffer (Qiagen) was performed following manufacturer's instructions. RNA was transcribed to cDNA using the Superscript III First-Strand Synthesis Supermix (Invitrogen). qPCR analysis was performed using the Platinum SYBR green qPCR Supermix-UDG kit (Invitrogen) using a ViiA 7 Real-time PCR instrument (Thermo Fisher Scientific). The expression of key hepatic genes, namely CYP3A4, PEPCK, CYP3A5, CYP2C9, CYP2D6, UGT1A1, HNF4a, HNF6, and ALB were studied. RPL19 was used as a reference gene for normalization. The expression levels in hydrogel spheroids were compared with their 2D hepatic counterparts and with two freshly isolated primary human hepatocyte (PHHs) from two patients as well as HepG2 cells (FIG. 15B). The results showed that the expression of CYP3A4 and CYP3A5 in hydrogel spheroids were comparable to those in PHHs. The expression of studied genes in hydrogel spheroids were mostly higher than 2D iPSC-3 derived hepatocytes showing the superiority of the 3D hydrogel spheroids over the 2D culture.

### Example 5. Effect of Various ECM Components in Differentiation of Hepatic Hydrogel Spheroids

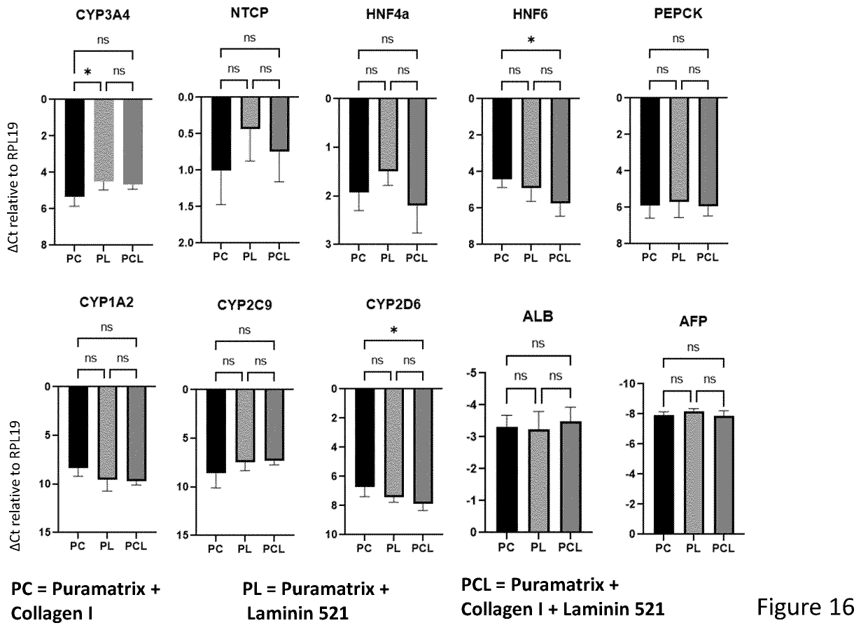

Hydrogel spheroids with hepatic cells described in example 4 were generated by mixing three combinations of ECMs into the hydrogel spheroids, namely Collagen I (PC), Laminin 521 (PL), and a mixture of both (PCL). Hydrogel spheroids were maintained for 14 days in hepatic medium before being collected and analysed by qPCR for the key hepatic genes, namely CYP3A4, NTCP, HNF4a, HNF6, PEPCK, CYP1A2, CYP2C9, CYP2D6, ALB, and AFP (FIG. 16). RPL19 was used as a reference gene for normalization. The comparison study showed relatively similar hepatic profile for the PC, PL, and PCL hydrogel spheroids, except that PL showed significantly higher expression of CYP3A4 compared to that in PC. Additionally, the expression of HNF6 and CYP2D6 were significantly higher in PC compared to those in PCL. The results showed that generation of hydrogel spheroids with hepatic cells using various combinations of ECMs is possible. This provides the great advantage of tailoring hydrogel spheroids according to the desired cell type(s) as mono- or co-culture with other cell types.

### Example 6. Potential of Hepatic Hydrogel Spheroids for Bulk Production and High-Throughput Assays

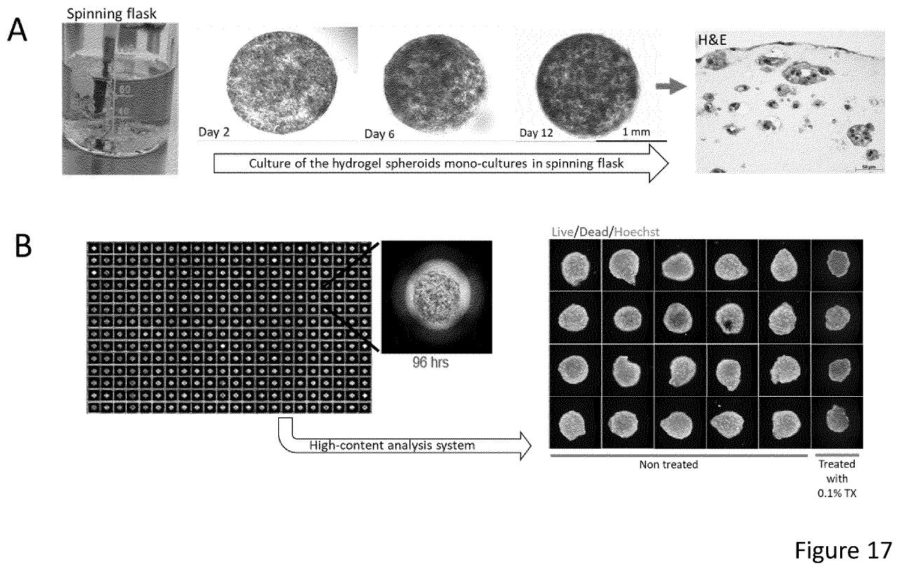

Spinning flask: Hydrogel spheroids with hepatic cells described in example 4 were generated in high quantities in a spinning flask and were maintained for 12 days in hepatic medium (FIG. 17A). Evaluating the hydrogel spheroids throughout the cultures showed that the hydrogel spheroids structure could remain intact through the course of culture in a bioreactor and iPSC-derived hepatic cells could maintain their growth as confirmed by H&E staining. This shows the potential of hydrogel spheroids for being mass produced for industrial or commercial applications.

High-throughput assays: HepG2 cells were cultured in 2D in DMEM (Invitrogen Cat #31885)+10% fetal bovine serum (FBS). Cells were detached at passage 12 by 0.05% trypsin and mixed (1×10⁷ cells/ml) with Puramatrix™+10% Bovine Collagen I (Gibco, Ref: A10644-01, Auckland, NZ) and 1 ul of the mix was pipetted in each well of a 384 well plate (Greiner microplate, Ref: 787979) to form the HepG2 hydrogel spheroids (one hydrogel spheroid per well). Hydrogel spheroids were maintained in HepG2 medium and the medium change was handled automatically by a robotic platform (NextGenQBio) for 19 days. Live/dead staining (Invitrogen, Ref:3224) was performed on the cell in the spheroids according the manufacturer's instruction. Prior to live/dead staining, a group of the hydrogel spheroids were treated with 0.1% Triton X for 15 minutes as control. Nuclei was stained by Hoechst (1:1000 dilution) for 15 minutes. The plate was imaged by a high-content analysis system (Operetta, PerkinElmer®). Results showed that majority of the HepG2 cells in non-treated hydrogel spheroids remained viable indicated by green colour, while hydrogel spheroids pre-treated with 0.1% Triton X showed minimal or no green colour and instead identified by a bright red colour indicating dead cells clearly distinguishing them from the non-treated hydrogel spheroids. This experiment highlights the potential of hydrogel spheroids to be used in a high-throughput set up specifically important for drug/compound screening and toxicity studies.

### Example 7. Co-Culturing the iPSC-3 Derived Hepatocytes with Pluripotent Stem Cell Derived Non-Parenchymal Cells

Endothelial differentiation: pluripotent stem cell-derived endothelial progenitor cells (termed as iETV2-SPi1) were differentiated according to the procedure already explained in the art (De Smedt, J., et. al., Cell Death Dis 12, 84 (2021). Briefly iETV2-SPi1 cells were maintained on Matrigel® (cat. 354277, Corning®) in E8 Flex medium (ThermoFisher). To start the differentiation, iETV2-SPi1 cells were passaged 1:6 and were maintained 1-2 days to reach ˜40% confluency. Cells were differentiated towards endothelial cells (ECs) by switching the medium to LDM supplemented with 5 μl/ml doxycycline. From day 2 onwards, 2.0% FBS was added to the medium. iETV2-SPi1 cells were passaged every 4-5 days using Tryple™ Express reagent (ThermoFisher, Cat #12605010) until their collection between day 8-12.

Macrophage differentiation: iPSC-3 was differentiated towards macrophages as described earlier in the art (B. van Wilgenburg, et. al., PloS One 8 (8) (2013), e71098) with some modifications. Briefly, iPSCs were resuspended at a final cell concentration of 1×10⁵ cells/mL in mTeSR™-1 medium (Stem Cell Technologies) supplemented with 50 ng/ml BMP-4, 20 ng/mL SCF, 50 ng/mL VEGF (all from Peprotech) and 1 μM Rock-inhibitor (Calbiochem) termed as EB medium. A volume of 100 μL of the suspension was seeded per well of 96-well ultra-low adherence plates (Greiner Bio-one), briefly centrifuged and then incubated at 37° C. and 5% CO2 for 4 days. About 50% of the medium was changed for fresh EB medium every day. On day 4, EBs were transferred and distributed in a 6 well plate culture, about 20 EBs in each well. The medium was switched to X-VIVO™15 (Lonza) supplemented with 50 ng/ml SCF, 50 ng/ml M-CSF, 50 ng/ml IL3, 50 ng/ml FLT3 and 5 ng/ml TPO (all from Peprotech), 2 mM Glutamax (Invitrogen), 100 U/mL penicillin, 100 μg/mL streptomycin (Invitrogen) and β-mercaptoethanol (0.055 mM, Invitrogen) until day 11. From day 11 onwards, the medium was switched to X-VIVO™15 supplemented with 50 ng/ml FLT3, 50 ng/ml M-CSF and 25 ng/ml GM-CSF (all from Peprotech), 2 mM Glutamax and 0.055 mM β-mercaptoethanol (Invitrogen) until the end of the differentiation. PSC-macrophage-like cells were collected from day 16 onward for encapsulation in hydrogel spheroids.

Stellate cell differentiation: the differentiation of hepatic stellate cells (HSCs) was performed as described earlier in the art [Coll et al. (2018) Cell Stem Cell 23(1),) 101-113 e7; Vallverdú et. al. (2021) Nat. Protoc. 16(5), 2542-2563). PSCs were maintained on Matrigel-coated plates until 70% confluency, when they were collected as single cells by Accutase™, and plated on Matrigel-coated plates at 2×10⁵ cells/ml in mTeSR medium supplemented with RevitaCell. Differentiation was started when cells reached 40% confluency and the medium was switched to LDM supplemented with different cytokine mixes as described in the art. On day 10, cells were dissociated with 0.05% Trypsin (Thermo Fisher Scientific) and re-plated with RevitaCell. On day 14, cells were collected by Accutase™ treatment and encapsulated in the hydrogels.

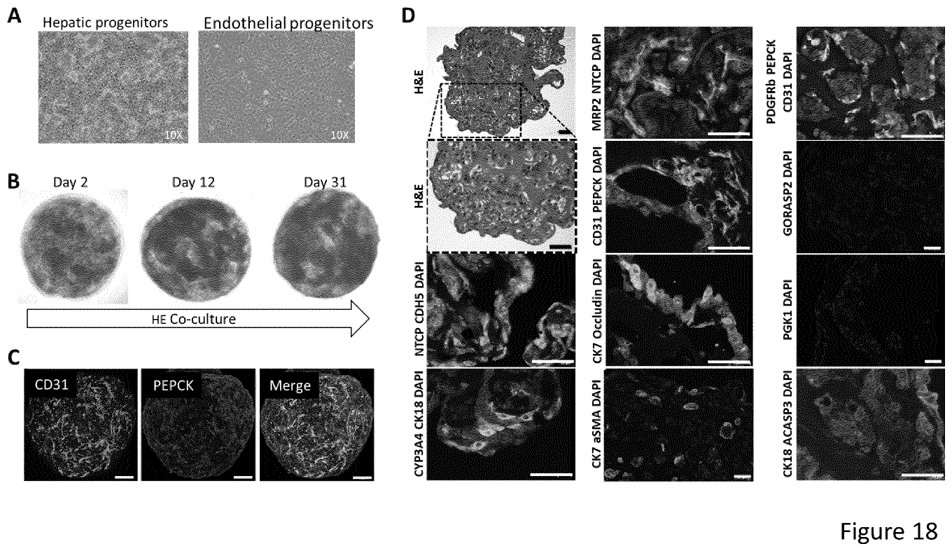

Hydrogel spheroids with hepatocytes and endothelial cells (HE): iPSC-3 derived hepatocyte progenitors described in example 4 were co-cultured with iETV2-SPi1 endothelial progenitor cells explained above at 2:1 ratio in hydrogel spheroids between day 8-12 (FIG. 18A). The co-culture of stem-cell derived hepatocytes and endothelial cells are termed as HE co-culture. Hydrogel spheroids with hepatocytes and endothelial cells were maintained in hepatic differentiation medium as described in example 4 for at least 32 days in hepatic medium supplemented with either 2% FBS or 1% Endothelial Cell Growth Supplement (ECGS, R&D, Cat #390599). The results revealed successful co-culture of hepatic and endothelial cells in hydrogel spheroid format. The bright field images showed integration and re-organisation of cells within the hydrogel spheroids during the course of culture (FIG. 18B). Results from confocal imaging showed the formation of an interconnected network of endothelial cells (indicated by CD31 positive cells) among the iPSC-derived hepatocytes (indicated by PEPCK positive cells) throughout the structure creating vascularised hydrogel spheroids (FIG. 18C). The H&E staining demonstrated a tissue-like structure within hydrogel spheroids recognised by dense areas and strong ECM deposited within the hydrogel spheroids. Images from immunohistochemistry showed the expression of hepatic markers namely NTCP, CYP3A4, MRP2, CK18, PEPCK, and Occludin demonstrating an organised and polarised structures developed by iPSC-derived hepatocyte in HE hydrogel spheroids. Additionally, the presence of endothelial cells and other non-parenchymal cells (NPCs) were confirmed by positive staining for CD31, CDH5, CK7, aSMA, and PDGFRb (FIG. 18D). This result demonstrates that the hydrogel spheroids with hepatocytes and endothelial cells minimally mimic the structure and natural organisation of the liver. Interestingly, we did not observe a necrotic area in the core of the hydrogel spheroids confirmed by GORASP2, PCK1 and the number of apoptotic cells confirmed by active CASP3 staining were negligible. The absence of necrotic core could be due to presence and properties of the hydrogel allowing the penetration and perfusion of the oxygen and nutrients to the deep areas within the hydrogel spheroids.

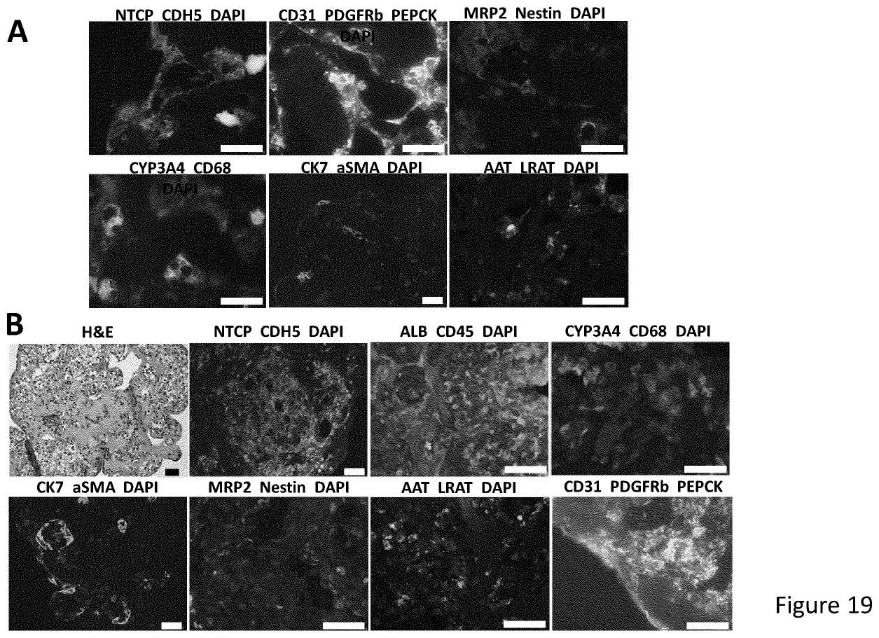

HME hydrogel spheroids (=hydrogel spheroids with hepatocytes macrophages, and endothelial cells): iPSC-3 derived hepatocyte progenitors described in example 4 were co-cultured with iPSC-3 derived macrophages, and iETV2-SPi1 endothelial progenitors in hydrogel spheroids. The hydrogel spheroids were cultured in 70% LDMAA maturation medium supplemented with 10 g/L glycine and 30% macrophage medium (including FLT3, M-CSF, and GM-CSF) as described above. Additionally, 1% ECGS was added to the culture medium. HME hydrogel spheroids were maintained for 17 days in culture before being collected for analysis. Immunohistochemistry results showed the positive expression for the markers NTCP, PEPCK, CYP3A4, and AAT positive cells confirming the presence of matured iPSC-hepatocytes in HME hydrogel spheroids (FIG. 19A). The presence of endothelial cells was confirmed by positive staining for the cells expressing CD31 and CDH5. Interestingly, in some locations these cells were nicely aligned along-side the lumenized areas representing the morphology of liver sinusoidal endothelial cells (LESC) observed in the liver (FIG. 19A, top raw). Notably, these cells were surrounded/supported by PDGFRb positive cells mimicking the function of stellate cells observed in the liver. The presence of macrophages was identified by the positive expression for CD68 marker. The presence of cholangiocytes were confirmed by detection of cells positively expressing CK7. Collectively, these results confirmed the successful co-culture of iPSC-hepatocytes, macrophages and endothelial cells in hydrogel spheroids.

HMES hydrogel spheroids (=hydrogel spheroids with hepatocytes macrophages, endothelial cells, and stellate cells): iPSC-3 derived hepatocyte progenitors described in example 4 were co-cultured with iPSC-3 derived macrophages, iETV2-SPi1 endothelial progenitors, and iPSC-3 derived hepatic stellate cells (HSC) in hydrogel spheroid format. The hydrogel spheroids were cultured in 70% LDMAA maturation medium supplemented with 10 g/L glycine and 30% macrophage medium (including FLT3, M-CSF, and GM-CSF) as described above. Additionally, the culture was supplemented with 1% ECGS, 5 μM retinol, and 100 μM palmitic acid. HMES hydrogel spheroids were maintained for 13 days in culture before being collected for analysis. Results from H&E staining showed the distribution of cell throughout the hydrogel spheroids creating a dense and tissue-like structure (FIG. 19B, top left). Immunostaining results confirmed the presence of all four cell types in HMES hydrogel spheroids. iPSC-hepatocytes were positively expressing NTCP, ALB, CYP3A4, MRP2, AAT, and PEPCK. Endothelial cells were identified by positive expression of CDH5, and CD31. Macrophages were identified by positive expression of CD45 and CD68 and stellate cells were positively expressing LRAT, nestin, and PDGFRb. Additionally, cholangiocytes were also identified in the hydrogel spheroids by positive expression of CK7. This data shows that the four cell type co-culture of hepatocyte together with non-parenchymal cells is possible in hydrogel spheroids.

1. A self-assembled peptide hydrogel spheroid, with a diameter of between 500 and 2500 μm, comprising cells encapsulated within said hydrogel.

2. The hydrogel spheroid according to claim 1, which is suspended in a cell culture medium.

3. The hydrogel spheroid according to claim 1 or 2, wherein the diameter of the spheroid is between 500 μm and 2000 μm or between 900 μm and 2000 μm.

4. The hydrogel spheroid, comprising between 1×10⁶ cells/ml and 5×10⁷ cells/ml.

5. The hydrogel spheroid according to any one of claims 1 to 4, wherein said cells are evenly distributed throughout the hydrogel spheroid.

6. The hydrogel spheroid according to any one of claims 1 to 4, wherein said cells occur as a layer of cells at the periphery of the hydrogel spheroid.

7. The hydrogel spheroid according to any one of claims 1 to 6, further comprising one or more extracellular matrix proteins or peptides.

8. The hydrogel spheroid according to any one of claims 1 to 7, wherein said cells are selected from the group consisting of hepatic cells including hepatocytes and non-parenchymal cells (NPCs), cardiac and muscle cells, endothelial cells, neural cells, pancreatic cells, osteocytes, chondrocytes, intestinal cells, fibroblasts, adipocytes, epithelial cells, pituitary cells, renal cells, lung cells, secretory cells, oral cells, germ cells, and cancer cell types or a mixture thereof.

9. The hydrogel spheroid according to any one of claims 1 to 8, wherein the cells are hepatic cells.

10. The hydrogel spheroid according to claim 8 or 9, wherein the hepatic cells are selected from the group consisting of primary hepatocytes, cells of a hepatic cell line, hepatic progenitor cells derived from mesenchymal or pluripotent stem cells, and hepatocytes differentiated from hepatic progenitor cells or mesenchymal or pluripotent stem cells.

11. The hydrogel spheroid according to claim 8, 9 or 10, wherein the hepatic cells are hepatocytes differentiated from pluripotent stem cells.

12. The hydrogel spheroid according to any of claim 8 to 11, wherein the hepatic cells express ALB, ASGR1 and AFP, NTCP, CYP3A4, CK18, MRP2, PEPCK, AAT1 and occludin.

13. The hydrogel spheroid according to claim 8, wherein the cardiac cells are selected from the group consisting of primary cardiomyocytes, cells of a cardiomyocyte cell line and cardiomyocytes differentiated from pluripotent stem cells.

14. The hydrogel spheroid according to claim 13, wherein the cardiac cells are primary cardiomyocytes.

15. The hydrogel spheroid according to any one of claims 1 to 14, wherein the cells are co-culture of hepatocytes and cardiomyocytes, typically beating cardiomyocytes.

16. An in vitro method of producing a hydrogel spheroid comprising cells, the method comprising:
a) mixing a suspension of cells with a self-assembling peptide, and
b) transferring an aliquot of the mixture obtained in step a) into an aqueous salt solution by applying a droplet of the mixture to the surface of the solution thereby forming a hydrogel spheroid comprising encapsulated cells,
wherein the droplet has a volume of between 0.1 and 20 μl.
and wherein the droplet comprises cells at a concentration of between 1×10⁵ to 5×10⁷ cells per ml solution.

17. The method according to claim 16, wherein the volume of the aliquot ranges from about 0.5 μl to about 20 μl.

18. The method according to claim 16 or 17, wherein said mixture comprises cells at a concentration of between 1×10⁶ cells per ml solution

19. The method according to any one of claims 16 to 18, wherein the cells are hepatic cells.

20. The method according to claim 19, wherein the hepatic cells are hepatocytes differentiated from pluripotent stem cells.

21. The method according to any one of claims 16 to 20, wherein the aqueous salt solution is a cell culture medium, preferably a differentiation medium for mesenchymal or pluripotent stem cells or progenitor cells.

22. The method according to any one of claims 16 to 21, further comprising cultivating the hydrogel spheroid in a cell culture medium for at least 4?, 8, 15, 20, 30 or 40 days.

23. The method according to any one of claims 16 to 22, wherein the hydrogel spheroids are suspended in said culture medium.

24. The method according to any one of claims 16 to 23, further comprising a step of freezing the hydrogel spheroids comprising cells.

25. Use of hydrogel spheroids comprising cells according to any one of claims 1 to 15, or prepared by the method according to any one of claims 16 to 24, for toxicity testing of compounds or pharmaceutical activity.

26. Hydrogel spheroids comprising cells according to any one of claims 1 to 15, or prepared by the method according to any one of claims 16 to 24, for use as a medicament.
